SEQUENCE LISTING (18 sequences)

SEQ ID NO: 1 - protein, 88 aa, Escherichia coli
MALLDFFLSRKKNTANIAKERLQIIVAERRRSDAEPHYLPQLRKDILEVICKYVQIDPEMVTVQLEQKDGDISILELNVTLPEAEELK

SEQ ID NO: 2 - protein, 231 aa, Escherichia coli
MSNTPIELKGSSFTLSVVHLHEAEPKVIHQALEDKIAQAPAFLKHAPVVLNVSALEDPVNWSAMHKAVSATGLRVIGVSGCKDAQLKAEIEKMGLPILTEGKEKAPRPAPTPQAPAQNTTPVTKTRLIDTPVRSGQRIYAPQCDLIVTSHVSAGAELIADGNIHVYGMMRGRALAGASGDRETQIFCTNLMAELVSIAGEYWLSDQIPAEFYGKAARLQLVENALTVQPLN

SEQ ID NO: 3 - protein, 270 aa, Escherichia coli
MARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVAKVLDDLKAMDFEFIVCDSPAGIETGALMALYFADEAIITTNPEVSSVRDSDRILGILASKSRRAENGEEPIKEHLLLTRYNPGRVSRGDMLSMEDVLEILRIKLVGVIPEDQSVLRASNQGEPVILDINADAGKAYADTVERLLGEERPFRFIEEEKKGFLKRLFGG

SEQ ID NO: 4 - protein, 164 aa, Bacillus subtilis
MPLTPNDIHNKTFTKSFRGYDEDEVNEFLAQVRKDYEIVLRKKTELEAKVNELDERIGHFANIEETLNKSILVAQEAAEDVKRNSQKEAKLIVREAEKNADRIINESLSKSRKIAMEIEELKKQSKVFRTRFQMLIEAQLDLLKNDDWDHLLEYEVDAVFEEKE

SEQ ID NO: 5 - DNA, 24 bp, Artificial Sequence
  Synthetic Construct
cccgcagctgagcttccgttatcg

SEQ ID NO: 6 - DNA, 24 bp, Artificial Sequence
  Synthetic Construct
cccgcagctgagcttccaatatcg

SEQ ID NO: 7 - DNA, 21 bp, Artificial Sequence
  Synthetic Construct
aacacgcgcaggctggttgtg

SEQ ID NO: 8 - DNA, 21 bp, Artificial Sequence
  Synthetic Construct
aacacgcgcaggctgattgtg

SEQ ID NO: 9 - skipped

SEQ ID NO: 10 - DNA, 59 bp, Artificial Sequence
  Synthetic Construct
ggatgagagggatgaaaaactcaaggcagagataactctgattacacgtcttgagcgat

SEQ ID NO: 11 - DNA, 59 bp, Artificial Sequence
  Synthetic Construct
ataaatgggagggtgacttgcctcaatataatccagactactgacatgggaattagcca

SEQ ID NO: 12 - DNA, 57 bp, Artificial Sequence
  Synthetic Construct
ctttgccataaaattctgctgggatttgatcactcagcattacacgtcttgagcgat

SEQ ID NO: 13 - DNA, 56 bp, Artificial Sequence
  Synthetic Construct
ctggcgatgattaatagctaattgagtaaggccaggactgacatgggaattagcca

SEQ ID NO: 14 - DNA, 59 bp, Artificial Sequence
  Synthetic Construct
acaagcgtttgaggaagcctttcttctcttcttcaatgaaattacacgtcttgagcgat

SEQ ID NO: 15 - DNA, 59 bp, Artificial Sequence
  Synthetic Construct
caaggaatttctatggcacgcattattgttgttacttcggctgacatgggaattagcca

SEQ ID NO: 16 - DNA, 59 bp, Artificial Sequence
  Synthetic Construct
ttgagtaaggccaggatgtcaaacacgccaatcgagcttttgtgtaggctggagctgct

SEQ ID NO: 17 - DNA, 60 bp, Artificial Sequence
  Synthetic Construct
ctttttgctcaagctggacggtaaccatttccggatcgatcggctgacatgggaattagc

SEQ ID NO: 18 - DNA, 3882 bp, Artificial Sequence
  Synthetic Construct
tcttgtgcaatgtaacatcagagattttgagacacaacgtggctttgttgaataaatcgaacttttgctgagttgaaggatcagcagtgctcttgtgggtccgattcgccagatgataaggaacagagacgggaccaaaacgaaaaaaggcccccctttcgggaggcctcttttctggaatttggtaccgaggcattcagtctgcctgctttcgcaggaacgccggaatgtccagataatccggctctttggctgtctgaggagcattatcattaacaactttggcgactggtttctgctcctgcgtaaggggcgccataccatgttgttgatagcggtccatcacaggctgctgaacctgtttattagtcactaaggtaatttctggtcttttatccatgccgattcccgtggcgacaaccgttacgcgcagttcatcattcatgtcagggtctaaggaggtacctataactacggtcgcgttgtctgaggcaaatgctctaatggtgtttcccacagtctcgaactcatctaaccgaagatcgaaaccggcagtaatatttacaagaacaccgcgggcgccggacaaatcaatatcctctaataacggtgaagatatagccatctcagcagcttcttctgcgcgatcctccccagatgccacgcctgagcccatcatggcatagcccatctctgacatcactgtcctcacatccgcaaaatctacgttcataagcccaggtcgggtgatcaattcggctataccctgaaccgcgcctttcagaacatcattagccgcaccgaacgcatccagcagtgagattcctcgccccaggactttaagcagtttatcatttgggatcgtgatcaatgaatcaacatgtttagaaagctccgtgatgccctgctccgcaaatgccatacgtttcttaccttcaaagttgaacggtttggtaactacggcaacggtcagaatacccagatctttggccacctccgccacaaccggcgccgcgcccgtccccgtgccgccgcccatgcctgcggcgataaaaaccatatctgccccttccaatgcggctctcaaggcgtcgcgatcctcatccgccgcgttgcgtcctacttccggattcgccccagcacccaaacctttggtgatgcctgacccgatctgaatagtttgacctacggcggttttacggagtgcctgagcatctgtgttcactgcaaagaattccacaccctcaatgcgttctctaaccatatgctcaacagcgttgccgccaccaccgccgactcctatgactttaataaccgcatcgttggtaagttccattggctcaaacatctgatcctccttagtatagtgacctcttaaacaaaattatttgtagaggcgctttcgtcctcacggactcatcagaacgggaagcacacccgttgagcgctttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaatcagagaatataaaaagccagattattaatccggcttttttattatttcgtctcggtggaagggctcggagttgccaggatacatagattaccacccgtagaaaagatcaaaagatcttcttgagatcctttttttctgcgcgtaatctgctacttgcaaacaaaaaaaccaccgctaccagcggtggtttgtttgccggatcaagagctaccaactctttttccgaaggtaactggctttagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatttccgctactgaactactcgatgtcagagccagcgtcttgcataaaaaaacccgccgaagcgggtttttacgttattattagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgaggcggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatattttta

(19) United States
(12) Patent Application Publication
(10) Pub. No.: US 2023/0242867 A1
(43) Pub. Date: Aug. 3, 2023

(54) METHODS FOR MANUFACTURING ADAS

(71) Applicant: Flagship Pioneering Innovations VI, LLC, Cambridge, MA (US)

(72) Inventors: Adam Barclay FISHER, Cambridge, MA (US); Troy Patrick HUBBARD, Somerville, MA (US); Caitlin Nicole SPAULDING, Boston, MA (US); Maier Steve Avendaño AMADO, Cambridge, MA (US)

(73) Assignee: Flagship Pioneering Innovations VI, LLC, Cambridge, MA (US)

(21) Appl. No.: 18/011,014

(22) PCT Filed: Jun. 17, 2021
(86) PCT No.: PCT/US2021/037759

§ 371 Date: Dec. 16, 2022

Related U.S. Application Data
(60) Provisional application No. 63/040,459, filed on Jun. 17, 2020.

(51) Int. Cl. C12N 1/20; C12N 1/02

(52) U.S. Cl. CPC C12N 1/20; C12N 1/02; C12N 2523/00

(57) ABSTRACT

The invention provides methods for manufacturing purified preparations of achromosomal dynamic active systems (ADAS), including highly active ADAS. These ADAS provided by the invention can be obtained by a variety of means. Various associated methods of making and using these ADAS are provided.

### CROSS-REFERENCES TO RELATED APPLICATIONS

This application claims priority to U.S. Patent Application No. 63/040,459, filed on Jun. 17, 2020, the entire contents of which are incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 15, 2021, is named 51296-042WO2_Sequence_Listing_6_15_21_ST25 and is 14,470 bytes in size.

### FIELD OF THE INVENTION

Provided herein are methods for manufacturing purified preparations of achromosomal dynamic active systems (ADAS), including highly active ADAS.

### BACKGROUND

A need exists for delivery vectors capable of targeting cells and delivering biological agents, compositions containing such delivery vectors, and associated methods of delivering said vectors to cells, thereby modulating biological systems including animal, plant, and insect cells, tissues, and organisms. In particular, there is a need for methods of separating delivery vectors from the parent cell lines from which they are produced.

### SUMMARY OF THE INVENTION

In one aspect, the disclosure features a method for producing an achromosomal dynamic active system (ADAS) preparation, the method comprising (a) providing a preparation comprising a plurality of ADAS and a plurality of parent bacterial cells and (b) exposing the preparation to a culture medium and a growth-selective agent under growth-promoting conditions for the parent bacterial cells, wherein the growth-selective agent reduces viability or inhibits cell division of the growing parent bacterial cells, thereby producing an ADAS preparation that is substantially enriched in ADAS.

In some embodiments, the preparation of step (a) has been concentrated relative to a culture from which the plurality of ADAS and plurality of parent bacterial cells are derived. In some embodiments, the preparation of step (a) has been concentrated by at least 20-fold, at least 50-fold, or at least 100-fold.

In some embodiments, the growth-selective agent is an agent that is toxic to parent bacterial cells.

In some embodiments, the agent that is toxic to parent bacterial cells is an antibiotic. In some aspects, the antibiotic is a beta lactam, ceftriaxone, kanamycin, carbenicillin, gentamicin, or ciprofloxacin.

In some embodiments, the agent that is toxic to parent bacterial cells is a chemical. In some embodiments, the chemical is sodium chloride, sodium hydroxide, M hydrochloric acid, glucose, a plurality of cas-amino acids, or a plurality of D-amino acids.

In some embodiments, the growth-selective agent is an agent that increases the sensitivity to sedimentation of parent bacterial cells. In some embodiments, the growth-selective agent induces a filamentous morphology in parent bacterial cells. In some embodiments, the sedimentation is performed by low-speed centrifugation.

In some embodiments, the growth-selective agent is an agent that interferes with growth of a bacterial cell wall.

In some embodiments, step (b) further comprises providing an agent that promotes the growth of parent bacterial cells.

In some embodiments, the exposing comprises incubating the preparation for at least one hour. In some embodiments, the incubating is performed at a temperature of between 4° C. and 42° C.

In some embodiments, the exposure to the culture medium precedes the exposure to the growth-selective agent.

In some embodiments, the preparation of step (a) is a pellet produced by a process comprising providing a supernatant of a culture comprising a plurality of ADAS and a plurality of parent bacterial cells, wherein the supernatant is produced by low-speed centrifugation of the culture, and subjecting the supernatant to high-speed centrifugation, thereby producing the pellet.

In some embodiments, step (b) comprises resuspending the pellet in the culture medium.

In some embodiments, the parent bacterial cells are derived from a culture at a stationary phase of growth. In some embodiments, the parent bacterial cells are senescent.

In some embodiments, the culture from which the plurality of ADAS and plurality of parent bacterial cells are derived has a volume of at least 1 L. In some embodiments, the culture has a volume of at least 100 L.

In some embodiments, the ADAS are derived from the parent bacterial cells.

In some embodiments, the method further comprises subjecting the ADAS preparation of step (b) to low-speed centrifugation, wherein the supernatant comprises the ADAS preparation.

In some embodiments, the ADAS preparation is substantially free of parent bacterial cells.

In some embodiments, the method further comprises concentrating the substantially enriched ADAS preparation.

In some embodiments, the method does not comprise contacting the parent cells with a nuclease.

In another aspect, the disclosure features an achromosomal dynamic active system (ADAS) preparation produced by any of the methods described herein, wherein the ratio of ADAS to parent cells in the preparation is greater than at least one of 1,000:1, 10,000:1, 100,000:1, 500,000:1, and 1,000,000:1.

In some embodiments of any of the above aspects, the growth-selective agent is present at a level less than at least one of 80 ng/ml, 70 ng/ml, 60 ng/ml, 50 ng/ml, 40, ng/ml, 30 ng/ml, 20 ng/ml, 10 ng/ml, 5 ng/ml, and 1 ng/ml following step (b) of the method.

Other features and advantages of the invention will be apparent from the following Detailed Description and the Claims.

### DETAILED DESCRIPTION OF THE INVENTION

### I. Definitions

As used herein, the term “growth-selective agent” refers to an agent that reduces viability (e.g., kills) or inhibits cell division of a growing bacterial cell, e.g., a growing bacterial cell, but does not reduce viability of or cause filamentation in an ADAS. Exemplary growth-selective agents include antibiotics, e.g., beta-lactam antibiotics, ceftriaxone, kanamycin, carbenicillin, gentamicin, or ciprofloxacin; chemicals, e.g., sodium hydroxide, M hydrochloric acid, glucose, a plurality of cas-amino acids, or a plurality of D-amino acids. In some embodiments, the growth-selective agent increases the sensitivity to sedimentation of a cell (e.g., sedimentation by low-speed centrifugation), e.g., induces a filamentous morphology in the cell. In some examples, the growth-selective agent is an agent that interferes with growth of a bacterial cell wall.

The term “growth-promoting conditions,” as used herein, refers to any condition that is permissive for bacterial growth or encourages activation of metabolism. An “agent that promotes the growth of parent bacterial cells” includes any agent that improves or allows for bacterial cell growth or division. Growth-promoting conditions may differ depending upon a bacterial strain. For example, growth-promoting conditions for an auxotrophic bacteria would require the missing nutrient (for example, amino acid). That nutrient would constitute an agent that promotes growth for that auxotrophic organism.

As used herein, the term “achromosomal dynamic system” or “ADAS” refers to a genome-free, non-replicating, enclosed membrane system comprising at least one membrane and having an interior volume suitable for containing a cargo (e.g., one or more of a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)). In some embodiments, ADAS are minicells or modified minicells derived from a parent bacterial cell (e.g., a gram-negative or a gram-positive bacterial cell). In other aspects, ADAS are substantially similar in size to the parent cell. ADAS may be derived from parent bacteria using any suitable method, e.g., genetic manipulation of the parent cell or exposure to a culture or condition that increases the likelihood of formation of bacterial minicells. Exemplary methods for making ADAS are those that disrupt the cell division machinery of the parent cell. In some embodiments, ADAS may comprise one or more endogenous or heterologous features of the parent cell surface, e.g., cell walls, cell wall modifications, flagella, or pilli, and/or one or more endogenous or heterologous features of the interior volume of the parent cell, e.g., nucleic acids, plasmids, proteins, small molecules, transcription machinery, or translation machinery. In other embodiments, ADAS may lack one or more features of the parent cell. In still other embodiments, ADAS may be loaded or otherwise modified with a feature not comprised by the parent cell.

As used herein, the term “highly active ADAS” refers to an ADAS having high work potential, e.g. an ADAS having the capability to do a significant amount of useful work. Work may be metabolic work, including chemical synthesis (e.g., of proteins, nucleic acids, lipids, carbohydrates, polymers, or small molecules), chemical modification (e.g., of proteins, nucleic acids, lipids, carbohydrates, polymers or small molecules), or transport (e.g., import, export, or secretion, e.g., secretion by a bacterial secretion system (e.g., T3SS)) under suitable conditions. In certain embodiments, highly active ADAS begin with a large pool of energy, e.g., energy in the form of ATP. In other embodiments, ADAS have the capacity to take up or generate energy/ATP from another source. Highly active ADAS may be identified, e.g., by having increased ATP concentration, increased ability to generate ATP, increased ability to produce a protein, increased rate or amount of production of a protein, and/or increased responsiveness to a biological signal, e.g., induction of a promoter.

As used herein, the term “parent bacterial cell” refers to a cell (e.g., a gram-negative or a gram-positive bacterial cell) from which an ADAS is derived. Parent bacterial cells are typically viable bacterial cells. The term “viable bacterial cell” refers to a bacterial cell that contains a genome and is capable of cell division. Preferred parent bacterial cells are derived from any of the strains in Table 1.

An ADAS composition or preparation that is “substantially free of” parent bacterial cells and/or viable bacterial cells is defined herein as a composition having no more than 500, e.g., 400, 300, 200, 150, 100 or fewer colony-forming units (CFU) per mL. An ADAS composition that is substantially free of parent bacterial cells or viable bacterial cells may include fewer than 50, fewer than 25, fewer than 10, fewer than 5, fewer than 1, fewer than 0.1, or fewer than 0.001 CFU/mL. including no bacterial cells.

The term “cell division topological specificity factor” refers to a component of the cell division machinery in a bacterial species that is involved in the determination of the site of the septum and functions by restricting the location of other components of the cell division machinery, e.g., restricting the location of one or more Z-ring inhibition proteins. Exemplary cell division topological specificity factors include minE, which was first discovered in E. coli and has since been identified in a broad range of gram-negative bacterial species and gram-positive bacterial species (Rothfield et al., Nature Reviews Microbiology, 3: 959-968, 2005). minE functions by restricting the Z-ring inhibition proteins minC and minD to the poles of the cell. A second exemplary cell division topological specificity factor is DivIVA, which was first discovered in Bacillus subtilis (Rothfield et al., Nature Reviews Microbiology, 3: 959-968, 2005).

The term “Z-ring inhibition protein” refers to a component of the cell division machinery in a bacterial species that is involved in the determination of the site of the septum and functions by inhibiting the formation of a stable FtsZ ring or anchoring such a component to a membrane. The localization of Z-ring inhibition proteins may be modulated by cell division topological specificity factors, e.g., minE and DivIVA. Exemplary Z-ring inhibition proteins include minC and minD, which were first discovered in E. coli and have since been identified in a broad range of gram-negative bacterial species and gram-positive bacterial species (Rothfield et al., Nature Reviews Microbiology, 3: 959-968, 2005). In E. coli and in other species, minC, minD, and minE occur at the same genetic locus, which may be referred to as the “min operon”, the minCDE operon, or the min or minCDE genetic locus.

As used herein, the term “reduction in the level or activity of a cell topological specificity factor,” refers to an overall reduction of any of 5%, 10%, 20%, 30%, 40%, 50%, 60%, 70%, 80%, 90%, 95%, 96%, 97%, 98%, 99%, or greater, in the level or activity of the cell topological specificity factor (e.g., protein or nucleic acid (e.g., gene or mRNA)), detected by standard methods, as compared to the level in a reference sample (for example, an ADAS produced from a wild-type cell or a cell having a wild-type minCDE operon or wild-type div/VA gene), a reference cell (for example, a wild-type cell or a cell having a wild-type minC, minD, minE, div/VA, or minCDE gene or operon), a control sample, or a control cell. In some embodiments, a reduced level or activity refers to a decrease in the level or activity in the sample which is at least about 0.9×, 0.8×, 0.7×, 0.6×, 0.5×, 0.4×, 0.3×, 0.2×, 0.1×, 0.05×, or 0.01× the level or activity of the cell topological specificity factor in a reference sample, reference cell, control sample, or control cell.

As used herein, the term “percent identity” refers to percent (%) sequence identity with respect to a reference polynucleotide or polypeptide sequence following alignment by standard techniques.

Alignment for purposes of determining percent nucleic acid or amino acid sequence identity can be achieved in various ways that are within the capabilities of one of skill in the art, for example, using publicly available computer software such as BLAST, BLAST-2, PSI-BLAST, or Megalign software. Those skilled in the art can determine appropriate parameters for aligning sequences, including any algorithms needed to achieve maximal alignment over the full length of the sequences being compared.

For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleotides or amino acids in B. In some embodiments, sequence identity, for example, in homologues of MinE or DivIVA proteins will have at least about 40%, 50%, 60%, 70%, 80%, 85%, 90%, or even 95% or greater amino acid or nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% or greater amino acid sequence or nucleic acid identity, to a native sequence MinE (or minE) or DivIVA (or div/VA) sequence as disclosed herein.

The phrases “modulating a state of a cell” as used herein, refers to an observable change in the state (e.g., the transcriptome, proteome, epigenome, biological effect, or health or disease state) of the cell (e.g., an animal, plant, or insect cell) as measured using techniques and methods known in the art for such a measurement, e.g., methods to measure the level or expression of a protein, a transcript, an epigenetic mark, or to measure the increase or reduction of activity of a biological pathway. Modulating the state of the cell may result in a change of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In some embodiments, modulating the state of the cell involves increasing a parameter (e.g., the level or expression of a protein, a transcript, or activity of a biological pathway) of the cell. Increasing the state of the cell may result in an increase of the parameter by at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In other embodiments, modulating the state of involves decreasing a parameter (e.g., the level or expression of a protein, a transcript, or activity of a biological pathway) of the cell. Decreasing the state of the cell may result in a decrease of the parameter by at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration).

As used herein, the term “heterologous” means not native to a cell or composition in its naturally-occurring state. In some embodiments “heterologous” refers to a molecule; for example, a cargo or payload (e.g., a polypeptide, a nucleic acid such as a protein-encoding RNA or tRNA, or small molecules) or a structure (e.g., a plasmid or a gene-editing system) that is not found naturally in an ADAS or the parent bacteria from which it is produced (e.g., a gram-negative or gram positive bacterial cell).

### II. Purification of ADAS and ADAS Preparations

In some aspects, the disclosure features a method for producing an achromosomal dynamic active system (ADAS) preparation. The method may be used to purify any population of ADAS, e.g., any of the ADAS-producing strains described herein (e.g., described in Sections III and IV herein).

In some aspects, the method comprises (a) providing a preparation comprising a plurality of ADAS and a plurality of parent bacterial cells and (b) exposing the preparation to a culture medium and a growth-selective agent under growth-promoting conditions for the parent bacterial cells, wherein the growth-selective agent reduces viability or inhibits cell division of the growing parent bacterial cells, thereby producing an ADAS preparation that is substantially enriched in ADAS (e.g., enriched at least 10-fold, 20-fold, 50-fold, 100-fold, 200-fold, 500-fold, 1000-fold, 2000-fold, 3000-fold, 5000-fold, or more than 5000-fold relative to an untreated preparation or relative to a preparation treated using a control method).

The growth-selective agent may be any agent that reduces viability (e.g., kills) or inhibits cell division of a growing bacterial cell, e.g., a growing bacterial cell, but does not reduce viability of or cause filamentation in an ADAS.

In some embodiments, the growth-selective agent is an agent that is toxic to parent bacterial cells.

In some embodiments, the agent that is toxic to parent bacterial cells is an antibiotic. The antibiotic may be, e.g., a beta lactam, ceftriaxone, kanamycin, carbenicillin, gentamicin, or ciprofloxacin.

In some embodiments, the agent that is toxic to parent bacterial cells is a chemical. The chemical may be, e.g., sodium chloride, sodium hydroxide, M hydrochloric acid, glucose, a plurality of cas-amino acids, or a plurality of D-amino acids.

In some embodiments, the growth-selective agent is an agent that increases the sensitivity to sedimentation of parent bacterial cells, e.g., by inducing a filamentous morphology in parent bacterial cells. In some embodiments, the sedimentation is performed by low-speed centrifugation. The low-speed centrifugation may be, e.g., centrifugation at a speed of between about 1000×g and 8000×g for about 10 minutes to about 120 minutes. In some embodiments, centrifugation is performed at a temperature of about 4° C. to about 42° C.

In some embodiments, the growth-selective agent is an agent that interferes with growth of a bacterial cell wall.

In some embodiments, step (b) further comprises providing an agent that promotes the growth of parent bacterial cells.

In some embodiments, the exposing comprises incubating the preparation for at least 10 minutes, 20 minutes, 30 minutes, 40 minutes, 50 minutes, 1 hour, two hours, three hours, four hours, five hours, six hours, or more than six hours. In some embodiments, the exposing comprises incubating the preparation for at least one hour. In some embodiments, the incubating is performed at a temperature of between 4° C. and 42° C.

In some embodiments, the exposure to the culture medium precedes the exposure to the growth-selective agent.

In some embodiments, the preparation of step (a) has been concentrated relative to a culture from which the plurality of ADAS and plurality of parent bacterial cells are derived, e.g., has been concentrated by at least 2-fold, 5-fold, 10-fold, 20-fold, 50-fold, 100-fold, 250-fold, 500-fold, 1000-fold, 5000-fold, or 10,000-fold relative to the initial culture. In some embodiments, the preparation is concentrated about 20-fold. In some embodiments, the preparation is concentrated about 100-fold. The concentration may be performed using, e.g., centrifugation or tangential flow filtration (TFF).

In some embodiments, the preparation of step (a) is a pellet produced by a process comprising providing a supernatant of a culture comprising a plurality of ADAS and a plurality of parent bacterial cells, wherein the supernatant is produced by low-speed centrifugation of the culture, and subjecting the supernatant to high-speed centrifugation, thereby producing the pellet. The high-speed centrifugation may be, e.g., centrifugation at a speed of between about 10.000×g and 50,000×g for about 10 minutes to about 120 minutes. Alternatively, the pellet may be produced using a process comprising TFF. In some embodiments, step (b) comprises resuspending the pellet in the culture medium.

An exemplary process for concentrating an ADAS preparation containing parent cells using TFF comprises use of variable pore sizes, e.g. pore sizes of 500 kilodalton-1 micron.

In some embodiments, the parent bacterial cells are derived from a culture at a stationary phase of growth. In some embodiments, the parent bacterial cells are senescent.

In some embodiments, the culture from which the plurality of ADAS and plurality of parent bacterial cells are derived has a volume of at least 5 mL, 10 mL, 25 L, 50 mL, 100 mL, 200 mL, 300 mL, 400 mL, 500 mL, 1 L, 2 L, 3 L, 4 L, 5 L, 10 L, 20 L, 30 L, 40 L, 50 L, 60 L, 70 L, 80 L, 90 L, or 100 L. In some embodiments, the culture has a volume of more than 100 mL. In some embodiments, the culture has a volume of at least 1 L. In some embodiments, the culture has a volume of at least 100 L.

In some embodiments, the ADAS are derived from the parent bacterial cells, e.g., derived from the minicells using any of the methods for ADAS production described herein.

In some embodiments, the method further comprises subjecting the ADAS preparation of step (b) to low-speed centrifugation and/or TFF, wherein the supernatant comprises the ADAS preparation.

In some embodiments, the ADAS preparation is substantially free of parent bacterial cells, e.g., has no more than 500, e.g., 400, 300, 200, 150, 100 or fewer colony-forming units (CFU) per mL. An ADAS preparation that is substantially free of parent bacterial cells or viable bacterial cells may include fewer than 50, fewer than 25, fewer than 10, fewer than 5, fewer than 1, fewer than 0.1, or fewer than 0.001 CFU/mL. including no bacterial cells.

In some embodiments, the method further comprises concentrating the substantially enriched ADAS preparation, e.g., concentrating the ADAS preparation by at least 5-fold, 10-fold, 20-fold, 30-fold, 40-fold, 50-fold, 60-fold, 70-fold, 80-fold, 90-fold, or 100-fold. In some embodiments, the substantially enriched ADAS preparation is concentrated by more than 100-fold relative to a preparation that has not been concentrated. The concentration may be performed using, e.g., centrifugation or tangential flow filtration (TFF).

In some embodiments, the method does not comprise contacting the parent cells with an agent that degrades genetic material. In some embodiments, the method does not comprise contacting the parent cells with a nuclease.

An exemplary process for concentrating a purified ADAS preparation using TFF comprises use of variable pore sizes, e.g. pore sizes of 50 kilodalton-0.2 micron.

Purification separates ADAS from viable parent bacterial cells which contain a genome and may be larger. Additional methods for purification described herein include centrifugation, selective outgrowth, and buffer exchange/concentration processes.

Also provided herein are ADAS preparations made according to any of the methods described herein. For example, ADAS preparations herein may have a ratio of ADAS to parent cells that is greater than at least one of 1,000:1, 10,000:1, 100,000:1, 500,000:1, and 1,000,000:1. According to the methods described herein, in some embodiments, parent cell burden is reduced by more than 3,000-fold when compared to standard methods, thus producing a product of higher purity having an improved ratio of ADAS to parent cells. As a further benefit, reduced parent cell burden is achievable with less growth-selective agent, which not only reduces the manufacturing input costs but also reduces waste stream processing. For example, significant purity increases may be achieved with 5-fold, a 10-fold, a 15, fold, a 20-fold, or greater reduction in the amount of growth-selective agent used in the process. As a further benefit still, in some embodiments, ADAS preparations will have reduced growth-selective agent residue levels. By way of example, ADAS preparations produced by any of the methods herein may have a growth-selective agent residue level (e.g., a residue of an agent that is toxic to parent cells (e.g., an antibiotic, e.g., a beta lactam, ceftriaxone, kanamycin, carbenicillin, gentamicin, or ciprofloxacin)) that is less than at least one of 80 ng/ml, 70 ng/ml, 60 ng/ml, 50 ng/ml, 40, ng/ml, 30 ng/ml, 20 ng/ml, 10 ng/ml, 5 ng/ml, and 1 ng/ml. Growth-selective agents will vary from embodiment to embodiment, but exemplary growth-selective agents will include those that are toxic to parent bacterial cells, e.g. antibiotics. Exemplary antibiotics include those listed herein such as a beta lactam, ceftriaxone, kanamycin, carbenicillin, gentamicin, and ciprofloxacin. Even further still, in many embodiments, improved purity levels, e.g. reduction in parent cell burden, are achievable without the need for additional nucleases or genetic constructs to reduce parent cell number by degradation of the genetic material in the parent cells (sometimes also referred to as genetic suicide).

In some aspects, also provided herein are ADAS preparations, and methods of comparing such preparations, wherein the preparations are substantially free of parent bacterial cells and/or viable bacterial cells, e.g., have no more than 500, e.g., 400, 300, 200, 150, or 100 or fewer than 50, fewer than 25, fewer than 10, fewer than 5, fewer than 1, fewer than 0.1 colony-forming units (CFU) per mL. In some embodiments, an ADAS preparation that is substantially free of parent bacterial cells may include no bacterial cells.

Auxotrophic parental strains can be used to make ADAS provided by the invention. Such manufacturing methods are useful for purification of the ADAS. For example, following ADAS generation, parent bacterial cells may be removed by growth in media lacking the nutrient (for example, amino acid) necessary for viability of the parent bacterium. In some embodiments, an ADAS provided by the invention is derived from a parental strain auxotrophic for at least 1, 2, 3, 4, or more of: arginine (e.g., knockout in argA, such as strains JW2786-1 and NK5992), cysteine knockout in cysE (such as strains JW3582-2 and JM15), glutamine e.g., knockout in glnA (such as strains JW3841-1 and M5004), glycine e.g., knockout in glyA (such as strains JW2535-1 and AT2457), Histidine e.g., knockout in hisB (such as strains JW2004-1 and SB3930), isoleucine e.g., knockout in ilvA (such as strains JW3745-2 and AB1255), leucine e.g., knockout in leuB (such as strains JW5807-2 and CV514), lysine e.g., knockout in lysA (such as strains JW2806-1 and KL334), methionine e.g., knockout in metA (such as strains JW3973-1 and DL41), phenylalanine e.g., knockout in pheA (such as strains JW2580-1 and KA197), proline e.g., knockout in proA (such as strains JW0233-2 and NK5525), Serine e.g., knockout in serA (such as strains JW2880-1 and JC158), threonine e.g., knockout in thrC (such as strains JW0003-2 and Gif 41), tryptophan e.g., knockout in trpC (such as strains JW1254-2 and CAG18455), Tyrosine e.g., knockout in tyrA (such as strains JW2581-1 and N3087), Valine/Isoleucine/Leucine e.g., knockout in ilvd (such as strains JW5605-1 and CAG18431).

In certain embodiments, the methods include using a single, double, triple, or quadruple auxotrophic parental strain, optionally wherein said parental strain further includes a plasmid expressing a ftsZ.

### III. Compositions

A. ADAS and Highly Active ADAS

The invention is based, at least in part, on Applicant's discovery of achromosomal dynamic active systems (ADAS), including highly active ADAS, which are able to provide a wide array of functions in a large number of environments. An “ADAS” is a genome-free, non-replicating, enclosed membrane system comprising at least one membrane (in some embodiments, two membranes, where the two membranes are non-intersecting) and having an interior volume suitable for containing a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)). Accordingly, the disclosure herein is also directed to ADAS compositions, such as the preparations described herein as well as those preparations when combined with additional components, e.g. nucleic acids, plasmids, polypeptides, proteins, enzymes, amino acids, small molecules, gene editing systems, hormones, immune modulators, carbohydrates, lipids, organic particles, inorganic particles, ribonucleoprotein complexes (RNPs)), carriers, inert ingredients, formulation auxiliaries, etc. as described in more detail below.

In some embodiments, ADAS are minicells or modified minicells derived from a parent bacterial cell (e.g., a gram-negative or a gram-positive bacterial cell). ADAS may be derived from parent bacteria using any suitable method, e.g., genetic manipulation of the parent cell or exposure to a culture or condition that increases the likelihood of formation of bacterial minicells.

In some embodiments, an ADAS has a major axis cross section between about 100 nm-500 μm (e.g., in certain embodiments, about: 100-600 nm, such as 100-400 nm; or between about 0.5-10 μm, and 10-500 μm). In certain embodiments, an ADAS has a minor axis cross section between about: 0.001, 0.01, 0.1, 1, 2, 3, 4, 5, 6, 7, 8, 9, 10, 11, 12, 13, 14, 15, 20, 30, 40, 50, 60, 70, 80, 90, up to 100% of the major axis. In certain embodiments, an ADAS has an interior volume of between about: 0.001-1 μm³, 0.3-5 μm³, 5-4000 μm³, or 4000-50×10⁷ μm³. In some embodiments, the ADAS is substantially similar in size to the parent cell, e.g., has a size (e.g., interior volume, major axis cross-section, and/or minor axis cross section) that is about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the size of the parent cell, has a size that is identical to that of the parent cell, or has a size that is about 101%, 102%, 103%, 104%, 105%, 106%, 107%, 108%, 109%, or 110% of the size of the parent cell.

In some embodiments, the invention provides highly active ADAS. A “highly active” ADAS is an ADAS with high work potential, e.g. an ADAS having the capability to do a significant amount of useful work. Work may be defined as, e.g., metabolic work, including chemical synthesis (e.g., synthesis of proteins, nucleic acids, lipids, carbohydrates, polymers, or small molecules), chemical modification (e.g., modification of proteins, nucleic acids, lipids, carbohydrates, polymers or small molecules), or transport (e.g., import, export, or secretion) under suitable conditions. In some embodiments, highly active ADAS begin with a large pool of energy, e.g., energy in the form of adenosine triphosphate (ATP). In other embodiments, ADAS have the capacity to take up or generate energy (e.g., ATP) from another source.

The term “ADAS provided by the invention” encompasses all embodiments of ADAS described herein, including, in particular embodiments, highly active ADAS, the set of which can be referenced as “highly active ADAS provided by the invention”, which is a subset of the ADAS provided by the invention.

In one aspect, the invention provides a composition comprising a plurality of highly active achromosomal dynamic active systems (ADAS), wherein the ADAS have an initial ATP concentration of at least 1 mM and wherein the composition is substantially free of viable bacterial cells.

In another aspect, the invention provides a composition comprising a plurality of highly active achromosomal dynamic active systems (ADAS), wherein the ADAS have an initial ATP concentration of at least 3 mM and wherein the composition is substantially free of viable bacterial cells.

In some embodiments, a highly active ADAS has an initial ATP concentration of at least 1 nM, 1.1. nM, 1.2 nM, 1.3 nM, 1.4 mM, 1.5 mM, 1.6 mM, 2 mM, 2.5 mM, 3 nM, 3.5 nM, 4 mM, 5 mM, 10 mM, 20 mM, 30 mM, or 50 mM. ATP concentration can be evaluated by a variety of means including, in certain embodiments, a BacTiter-Glo™ assay (Promega) on lysed ADAS.

High activity may be additionally or alternatively assessed as the rate or amount of increase in ATP concentration in an ADAS over time. In some embodiments, the ATP concentration of an ADAS is increased by at least 50%, at least 60%, at least 75%, at least 100%, at least 150%, at least 200%, or more than 200% following incubation under suitable conditions, e.g., incubation at 37° C. for 12 hours. In certain embodiments, a highly active ADAS has a rate of ATP generation greater than about: 0.000001, 0.00001, 0.0001, 0.001, 0.01, 0.05, 0.1, 0.5, 1.0, 2, 3, 5, 10, 15, 20, 30, 40, 50, 75, 100, 200, 300, 500, 1000, 10000 ATP/sec/nm² for at least about: 1 hour, 2 hours, 3 hours, 4 hours, 5 hours, 6 hours, 12 hours, 1 day, 2 days, 4 days, 1 week, or two weeks.

In other aspects, high activity is assessed as a rate of decrease in ATP concentration over time. In some embodiments, ATP concentration may decrease less rapidly in ADAS that are highly active than in ADAS that are not highly active. In some embodiments, the drop in ATP concentration in an ADAS or an ADAS composition at 24 hours after preparation is less than about 50% (e.g., less than about: 45, 40, 35, 30, 25, 20, 15, 10, or 5%) compared to the initial ATP concentration (e.g., ATP per cell volume), e.g., as measured using a BacTiter-Glo™ assay (Promega).

High activity may be additionally or alternatively assessed as lifetime index of an ADAS. The lifetime index is calculated as the ratio of the rate of GFP production at 24 hours vs. 30 minutes. In some embodiments, a highly active ADAS has a lifetime index of greater than about: 0.13, 0.14, 0.15, 0.16, 0.18, 0.2, 0.25, 0.3, 0.35, 0.45, 0.5, 0.60, 0.70, 0.80, 0.90, 1.0 or more. In more particular embodiments, lifetime index is measured in an ADAS containing a functional GFP plasmid with a species-appropriate promoter in which GFP concentration is measured relative to number of ADAS, average number of plasmids per ADAS, and solution volume with a plate reader at 30 minutes and 24 hours.

In some aspects, the ADAS produces a protein, e.g., a heterologous protein. In some aspects, high activity is assessed as a rate, amount, or duration of production of a protein or a rate of induction of expression of the protein (e.g., responsiveness of an ADAS to a signal). For example, the ADAS may comprise a plasmid, the plasmid comprising an inducible promoter and a nucleotide sequence encoding the heterologous protein, wherein contacting the ADAS with an inducer of the inducible promoter under appropriate conditions results in production of the heterologous protein. In some aspects, the production of the heterologous protein is increased by at least 1.6-fold in an ADAS, e.g., a highly active ADAS, that has been contacted with the inducer relative to an ADAS that has not been contacted with the inducer. For example, in some embodiments, the production of the heterologous protein is increased by at least 1.5-fold, 1.75-fold, 2-fold, 3-fold, 4-fold, 5-fold, 10-fold, or more than 10-fold in an ADAS, e.g., a highly active ADAS, that has been contacted with the inducer. In some embodiments, the rate of production of the heterologous protein by a highly active ADAS reaches a target level within a particular duration following the contacting of the ADAS with the inducer, e.g., within 5 minutes, 10 minutes, 15 minutes, 30 minutes, 45 minutes, 1 hour, 2 hours, 3 hours, or more than 3 hours. In some embodiments, a protein (e.g., a heterologous protein) is produced at a rate of at least 0.1 femtograms per hour per highly active ADAS, e.g., at least 0.2 0.4, 0.6, 0.8, 1, 2, 4, 6, 8, 10, 25, 50, 100, 250, 500, 1000, 2000, 3000, or 3500 fg/hour per ADAS. In some embodiments, high activity of an ADAS is assessed as a duration for which a protein is produced. A highly active ADAS may produce a protein (e.g. a heterologous protein) for a duration of at least 2 hours, at least 4 hours, at least 8 hours, at least 12 hours, at least 24 hours, at least 48 hours, or longer than 48 hours.

B. ADAS and Highly Active ADAS Derived from Parent Bacteria Deficient in a Cell Division Topological Specificity Factor

ADAS may be derived from bacterial parent cells, as described herein.

In some aspects, the invention provides an ADAS and/or a composition comprising a plurality of ADAS derived from a parent bacterium having a reduction in a level, activity, or expression of a cell division topological specificity factor.

In some aspects, the invention provides a composition comprising a plurality of ADAS, wherein the ADAS do not comprise a cell division topological specificity factor and wherein the composition is substantially free of viable bacterial cells.

In some aspects, the invention provides a composition comprising a plurality of ADAS, the composition being substantially free of viable bacterial cells, and being produced by a process comprising: (a) making, providing, or obtaining a plurality of parent bacteria having a reduction in the level or activity of a cell division topological specificity factor; (b) exposing the parent bacterium to conditions allowing the formation of a minicell, thereby producing the highly active ADAS; and (c) separating the ADAS from the parent bacterium, thereby producing a composition that is substantially free of viable bacterial cells.

In some embodiments of the above aspects, the cell division topological specificity factor is a polypeptide having an amino acid sequence with at least 20% identity to an E. coli minE polypeptide (SEQ ID NO: 1), e.g., at least 30%, 40%, 50%, 60%, 70%, 80%, 90%, 95%, 98%, or 99% identity to SEQ ID NO: 1. In some embodiments, the cell division topological specificity factor comprises the amino acid sequence of SEQ ID NO: 1. In some embodiments, the cell division topological specificity factor is a minE polypeptide. Exemplary species having minE polypeptides are provided in Table 1 and in Rothfield et al., Nature Reviews Microbiology, 3: 959-968, 2005.

In some embodiments, the parent bacterium is E. coli and the minE polypeptide is E. coli minE. In other embodiments, the parent bacterium is Salmonella typhimurium and the minE polypeptide is S. typhimurium minE. In yet other embodiments, the parent bacterium is an Escherichia, Acinetobacter, Agrobacterium, Anabaena, Anaplasma, Aquifex, Azoarcus, Azospirillum, Azotobacter, Bartonella, Bordetella, Bradyrhizobium, Brucella, Buchnera, Burkholderia, Candidatus, Chromobacterium, Coxiella, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylobacterium, Methylococcus, Neisseria, Nitrosomonas, Nostoc, Photobacterium, Photorhabdus, Phyllobacterium, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Rhizobium, Rickettsia, Symbiobacterium, or Thermoanaerobacter bacterium and the cell division topological specificity factor is the endogenous minE or DivIVA of the parent bacterium.

In some embodiments of the above aspects, the cell division topological specificity factor is a polypeptide having an amino acid sequence with at least 20% identity to a Bacillus subtilis DivIVA polypeptide (SEQ ID NO: 4), e.g., at least 30%, 40%, 50%, 60%, 70%, 80%, 90%, 95%, 98%, or 99% identity to SEQ ID NO: 4. In some embodiments, the cell division topological specificity factor comprises the amino acid sequence of SEQ ID NO: 4. In some embodiments, the cell division topological specificity factor is a DivIVA polypeptide. Exemplary species having DivIVA polypeptides are provided in Table 1 and in Rothfield et al., Nature Reviews Microbiology, 3: 959-968, 2005. In some embodiments, the parent bacterium is Bacillus subtilis and the cell division topological specificity factor is B. subtilis DivIVA.

In some embodiments, the ADAS or parent bacterium having the reduction in a level or activity of the cell division topological specificity factor also has a reduction in a level of one or more Z-ring inhibition proteins.

In some embodiments, the Z ring inhibition protein is a polypeptide having an amino acid sequence with at least 20% identity to an E. coli minC polypeptide (SEQ ID NO: 2), e.g., at least 30%, 40%, 50%, 60%, 70%, 80%, 90%, 95%, 98%, or 99% identity to SEQ ID NO: 2. In some embodiments, the Z ring inhibition protein comprises the amino acid sequence of SEQ ID NO: 2. In some embodiments, the Z ring inhibition protein is a minC polypeptide.

In some embodiments, the Z ring inhibition protein is a polypeptide having an amino acid sequence with at least 20% identity to an E. coli minD polypeptide (SEQ ID NO: 3), e.g., at least 30%, 40%, 50%, 60%, 70%, 80%, 90%, 95%, 98%, or 99% identity to SEQ ID NO: 3. In some embodiments, the Z ring inhibition protein comprises the amino acid sequence of SEQ ID NO: 3. In some embodiments, the Z ring inhibition protein is a minD polypeptide.

In some embodiments, the ADAS or parent bacterium has a reduction in the level, activity, or expression of at least two Z-ring inhibition proteins. In some embodiments, the ADAS or parent bacterium has a reduction in expression of a minC polypeptide and a minD polypeptide. In some embodiments, the ADAS or parent bacterium has a reduction in expression of a minC polypeptide, a minD polypeptide, and a minE polypeptide, e.g., a deletion of the minCDE operon (ΔminCDE).

A reduction in the level, activity, or expression of a cell division topological specificity factor or a Z-ring inhibition protein, e.g., a reduction in an ADAS or a reduction in a parent bacterial cell, may be achieved using any suitable method. For example, in some embodiments, the reduction in the level or activity is caused by a loss-of-function mutation, e.g., a gene deletion. In some embodiments, the loss-of-function mutation is an inducible loss-of-function mutation and loss of function is induced by exposing the parent cell to an inducing condition, e.g., the inducible loss-of-function mutation is a temperature-sensitive mutation and wherein the inducing condition is a temperature condition.

In some embodiments, the parent cell has a deletion of the minCDE operon (ΔminCDE) or homologous operon.

C. ADAS Comprising a Cargo

In some embodiments, an ADAS provided by the invention includes a cargo contained in the interior of the ADAS. A cargo may be any moiety disposed in the interior of an ADAS (e.g., encapsulated by the ADAS) or conjugated to the surface of the ADAS. In some embodiments, the cargo comprises a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP) or a combination of the foregoing.

In some embodiments, the nucleic acid is a DNA, an RNA, or a plasmid. In some embodiments, the nucleic acid (e.g., DNA, RNA (e.g., mRNA, ASO, circular RNA, siRNA, shRNA, tRNA, dsRNA, or a combination thereof), or plasmid) encodes a protein. In some embodiments, the protein is transcribed and/or translated in the ADAS. In some embodiments, the nucleic acid inhibits translation of a protein or polypeptide, e.g., is an siRNA or an antisense oligonucleotide (ASO).

In some embodiments, the cargo is an agent that can modulate the microbiome of the target organism (e.g., a human, animal, plant, or insect microbiome), e.g., a polysaccharide, an amino acid, an anti-microbial agent (e.g., e.g., an anti-infective or antimicrobial peptide, protein, and/or natural product), a short chain fatty acid, or a combination thereof. In some examples, the agent that can modulate the host microbiome is a probiotic agent.

In some embodiments, the cargo is an enzyme. In some embodiments, the enzyme alters a substrate to produce a target product. In some embodiments, the substrate is present in the ADAS and the target product is produced in the ADAS. In other embodiments, the substrate is present in a target cell or environment to which the ADAS is delivered.

In certain embodiments, the cargo is modified for improved stability compared to an unmodified version of the cargo. “Stability” of a cargo is a unitless ratio of half-life of unmodified cargo and modified cargo half-life, as measured in the same environmental conditions. In some embodiments the environment is experimentally controlled, e.g., a simulated body fluid, RNAse free water, cell cytoplasm, extracellular space, or “ADAS plasm” (i.e., the content of the interior volume of an ADAS, e.g., after lysis). In some applications it is an agricultural environment, e.g., variable field soil, river water, or ocean water. In other embodiments, the environment is an actual or simulated: animal gut, animal skin, animal reproductive tract, animal respiratory tract, animal blood stream, or animal extracellular space. In certain embodiments, the ADAS does not substantially degrade the cargo.

In certain embodiments, the cargo comprises a protein. In certain embodiments, the protein has stability greater than about: 1.01, 1.1, 10, 100, 1000, 10000, 100000, 100000, 10000000 in cell cytoplasm or other environments. The protein can be any protein, including growth factors; enzymes; hormones; immune-modulatory proteins; antibiotic proteins, such as antibacterial, antifungal, insecticide, proteins, etc.; targeting agents, such as antibodies or nanobodies, etc. In some embodiments, the protein is a hormone, e.g., paracrine, endocrine, autocrine.

In some embodiments, the cargo comprises a plant hormone, such as abscisic acid, auxin, cytokinin, ethylene, gibberellin, or a combination thereof.

In some embodiments, the cargo is an anti-inflammatory agent, e.g., a cytokine (e.g., a heterologously expressed anti-inflammatory cytokine or mutein thereof, e.g., IL-10, TGF-Beta, IL-22, IL-2) or an antibody (e.g., an antibody or antibody fragment targeting tumor necrosis factor (TNF) (e.g., an anti-TNF antibody); an antibody or antibody fragment targeting IL-12 (e.g., an anti-IL-12 antibody); or an antibody or antibody fragment targeting IL-23 (e.g., an anti-IL-23 antibody).

In certain embodiments, the cargo is an immune modulator. Immune modulators include, e.g., immune stimulators; checkpoint inhibitors (e.g., inhibitors of PD-1, PD-L1, or CTLA-4); chemotherapeutic agents; immune suppressors; antigens; super antigens; and small molecules (e.g., cyclosporine A, cyclic dinucleotides (CDNs), or STING agonists (e.g., MK-1454)). In some embodiments, the immune modulator is a moiety that induces tolerance in a subject, e.g., an allergen, a self-antigen (e.g., a disease-associated self-antigen), or a microbe-specific antigen. In some embodiments, the immune modulator is a vaccine, e.g., an antigen from a pathogen (e.g., a virus (e.g., a viral envelope protein) or a bacteria). In some embodiments, the pathogen is a coronavirus, e.g., SARS-Ooh′-2. In some embodiments, the antigen is a cancer neo-antigen. In some embodiments, the cargo an adjuvant, e.g., an immunomodulatory molecule or a molecule that alters the compartmentalization, presentation, or profile of one or more co-stimulatory molecules associated with a vaccine antigen. In some examples, the adjuvant is an activator of an immune pathway upstream of a desired immune response (e.g., an activator of an innate immune pathway upstream of an adaptive immune response). In other examples, the adjuvant enhances the presentation of an antigen on an immune cell or immune moiety (e.g., MHC class 1) in the target organism. In some examples, the adjuvant is listeriolysin O (LLO). In some embodiments, an ADAS comprises an antigen and one or more adjuvants.

In some embodiments, the cargo is an agent for treatment or prevention of a cancer, e.g., an agent that decreases the likelihood that a patient will develop a cancer or an agent that treats a cancer (e.g., an agent that increases progression-free survival and/or overall survival in an individual having a cancer).

Agents for the prevention of cancer include, but are not limited to anti-inflammatory agents and growth inhibitors. Agents for the treatment of cancer (e.g., a solid tumor cancer) include, but are not limited to anti-inflammatory agents, growth inhibitors, chemotherapy agents, immunotherapy agents, anti-cancer antibodies or antibody fragments (e.g., antibodies or antibody fragments targeting cancer antigens (e.g., cancer neo-antigens)), cancer vaccines (e.g., vaccines comprising a cancer neo-antigen), agents that induce autophagy (e.g., activators such as listeria-lysin-o), cytotoxins, inflammasome inhibiting agents, immune checkpoint inhibitors (e.g., inhibitors of PD-1, PD-L1, or CTLA-4), transcription factor inhibitors, and agents that disrupt the cytoskeleton.

In some embodiments, the cargo is an enzyme. The enzyme may be an enzyme that performs a catalytic activity in a target cell or organism (e.g., in a human, animal, plant, or insect). In some embodiments, the catalytic activity is extracellular matrix (ECM) digestion (e.g., the enzyme is hyaluronidase and the catalytic activity is ECM digestion) or removal of toxins. In some embodiments, the enzyme is an enzyme replacement therapy, e.g., is phenylalanine hydroxylase. In some embodiments, the enzyme is a UDP-glucuronosyltransferase. In some embodiments, the enzyme has hepatic enzymatic activity (e.g., porphobilinogen deaminase (PBGD), e.g., human PBGD (hPBGD)). In some embodiments, the enzyme is a protease, oxidoreductase, or a combination thereof.

In some embodiments, the enzyme alters a substrate to produce a target product. In some embodiments, the substrate is present in the ADAS and the target product is produced in the ADAS. In other embodiments, the substrate is present in a target cell or environment to which the ADAS is delivered. In some embodiments, the enzyme is diadenylate cyclase A, the substrate is ATP, and the target product is cyclic-di-AMP.

In some embodiments, the enzyme is chemically conjugated to the ADAS membrane, optionally via a linker to the exterior membrane.

Alternatively, the cargo may be a nucleic acid that encodes any of the enzymes described herein.

In some embodiments, the cargo is an agent that activates or inhibits autophagic processes (e.g. an activator such as listeria-lysin-o or an inhibitor such as IcsB).

In some embodiments, the cargo is an anti-infective agent, e.g., an anti-microbial agent, e.g., an anti-infective or antimicrobial peptide, protein, and/or natural product.

In some embodiments, the cargo is a protein that modulates host transcriptional response e.g., a transcription factor; a protein that promotes host cell growth, e.g., a growth factor; or a protein that inhibits protein function, e.g., a nanobody. In some embodiments, the transcription factor is a human transcription factor.

For ADAS comprising cargo, in some embodiments, the cargo is an RNA, such as circular RNA, mRNA, siRNA, shRNA, ASO, tRNA, dsRNA, or a combination thereof. In certain embodiments, the RNA has stability greater than about: 1.01, 1.1, 10, 100, 1000, 10000, 100000, 100000, 10000000, e.g., in ADAS plasm. The RNA cargo can be stabilized, in certain embodiments, e.g., with an appended step-loop structure, such as a tRNA scaffold. For example, non-human tRNALys3 and E. coli tRNAMet (Nat. Methods, Ponchon 2007). Both have been well characterized and expressed recombinantly. However, a variety of other types could be used as well, such as aptamers, lncRNA, ribozymes, etc. RNA can also be stabilized where the ADAS is obtained from a parental strain null (or hypomorphic) for one or more ribonucleases.

In some particular embodiments, the RNA is a protein-coding mRNA. In more particular embodiments, the protein-coding mRNA encodes an enzyme (e.g., and enzyme that imparts hepatic enzymatic activity, such as human PBGD (hPBGD) mRNA) or an antigen, e.g., that elicits an immune response (such as eliciting a potent and durable neutralizing antibody titer), such as mRNA encoding CMV glycoproteins gB and/or pentameric complex (PC)). In certain particular embodiments, the RNA is a small non-coding RNA, such as shRNA, ASO, tRNA, dsRNA, or a combination thereof.

In certain embodiments, the ADAS provided by the invention includes cargo comprising at least one component of a gene editing system. Components of a “gene editing system” include (or encode) proteins (or nucleic acids encoding said proteins) that can, with suitable associated nucleic acids, modify a DNA sequence of interest, such as a genomic DNA sequence, whether e.g., by insertion or deletion of a sequence of interest, or by altering the methylation state of a sequence of interest, as well as nucleic acids associated with the function of such proteins, e.g., guide RNAs. Exemplary gene editing systems include those based on a Cas system, such as Cas9, Cpf1 or other RNA-targeted systems with their companion RNA (e.g., sequence-complementary CRISPR guide RNA), as well as Zinc finger nucleases and TAL-effectors conjugated to nucleases.

Other embodiments of ADAS provided by the invention include DNA as the cargo, including as a plasmid, optionally wherein the DNA comprises a protein-coding sequence. Exemplary DNA cargo includes, in certain embodiments, a plasmid encoding an RNA sequence of interest (see examples above), e.g., which may be flanked on each side by an tRNA insert. Various DNA cargo are encompassed by the invention, including: ADAS producing (e.g., driving FTZ overexpression, genome degrading exonucleases); longevity plasmids (ATP synthase expressing, rhodopsin-expressing); those expressing stabilized non-coding RNA, tRNA, lncRNA; expressing secretion system tag proteins, NleE2 effector domain and localization tag; secretion systems T3/4SS, TSSS, T6SS; logic circuits, conditionally expressed secretion systems; and combinations thereof. In some embodiments, a logic circuit includes inducible expression or suppression cassettes, such as IPTG-inducible Plac promoter and the hrpR portion of the AND gate, and, for example, the heat-induced promoter pL (from phage lambda, which is usually suppressed by a thermolabile protein) and the hrpS portion of the AND gate. To engineer an OR gate, a sytem described by Rosado et al., PLoS Genetics, 2018 can be used. Briefly, a cis-repressed mRNA coding for RFP under a constitutive promoter can be used. The repression can then be removed in the presence of RAJ11 sRNA. Plasmids containing the IPTG-inducible promoter PLac and heat-induced promoter pL, both of which induce the expression of RAJ11 sRNA, can then be used. The output would then be RFP expression, which is seen in response to either input. These systems can be adapted to a variety of sensor-type functions.

ADAS provided by the invention, in some embodiments, include a transporter in the membrane. In certain embodiments, the transporter is specific for glucose, sodium, potassium, a metal ion, an anionic solute, a cationic solute, or water.

In some embodiments, the membrane of an ADAS provided by the invention comprises an enzyme. In particular embodiments, the enzyme is a protease, oxidoreductase, or a combination thereof. In some embodiments, the enzyme is chemically conjugated to the ADAS membrane, optionally via a linker to the exterior membrane.

D. ADAS Comprising a Secretion System

In certain embodiments, an ADAS provided by the invention comprises a bacterial secretion system (e.g., an endogenous bacterial secretion system or a heterologous secretion system). A “bacterial secretion system” is a protein, or protein complex, that can export a cargo from the cytoplasm of a bacterial cell (or, for example, an ADAS derived therefrom) into: the extracellular space, the periplasmic space of a gram-negative bacterium, or the intracellular space of another cell. In some embodiments, the bacterial secretion system works by an active (e.g., ATP-dependent or PMF-dependent) process, and in certain embodiments the bacterial secretion system comprises a tube or a spike spanning the host cell (or ADAS) to a target cell. In other embodiments the bacterial secretion system is a transmembrane channel. Exemplary bacterial secretion systems include T3SS and T4SS (and T3/T4SS, as defined, below), which are tube-containing structures where the cargo traverses through the inside of a protein tube and T6SS, which carries the cargo at the end of a spike. Other exemplary bacterial secretion systems include T1SS, T2SS, TSSS, T7SS, Sec, and Tat, which are transmembrane.

In some embodiments, the heterologous secretion system is a T3SS.

In some embodiments, the ADAS comprises a cargo, wherein the cargo comprises a moiety that directs export by the bacterial secretion system, e.g., in some embodiments the moiety is Pho/D, Tat, or a synthetic peptide signal.

In certain embodiments, the ADAS provided by the invention are two-membrane ADAS. In more particular embodiments the two-membrane ADAS further comprises a bacterial secretion system. In still more particular embodiments, the bacterial secretion system is selected from T3SS, T4SS, T3/4SS, or T6SS, optionally wherein the T3SS, T4SS, T3/4SS, or T6SS have an attenuated or non-functional effector that does not affect fitness of a target cell.

ADAS provided by the invention, in some embodiments, include a bacterial secretion system.

In some embodiments, the bacterial secretion system is capable of exporting a cargo across the ADAS outer membrane into a target cell, such as an animal, fungal, bacterial, or plant cell, such as T3SS, T4SS, T3/T4SS, or T6SS.

In more particular embodiments, the bacterial secretion system is a T3/4SS. A “T3/4SS” is a secretion system based on T3SS or T4SS, including hybrid systems as well as unmodified versions, which forms a protein tube between a bacterium (or ADAS) and a target cell, connecting the two and delivering one or more effectors. The target cell can be an animal, plant, fungi, or bacteria. In some embodiments a T3/4SS includes an effector, which may be a modified effector. Examples of T3SS systems include the Salmonella SPI-1 system, the EHEC coli ETT1 system, the Xanthamonas Citri/Campestri T3SS system, and the Pseudomonas syringae T3SS system. Examples of T455 systems include the Agrobacterium Ti plasmid system, Helicobacter pylori T4SS. In certain embodimnets, the T3/4SS has modified effector function, e.g., an effector selected from SopD2, SopE, Bop, Map, Tir, EspB, EspF, NleC, NleH2, or NleE2. In more particular embodiments, the modified effector function is for intracellular targeting such as translocation into the nucleus, golgi, mitochondria, actin, microvilli, ZO-1, microtubules, or cytoplasm. In still more particular embodiments, the modified effector function is nuclear targeting based on NleE2 derived from E. Coli. In other particular embodiments, the modified effector function is for filopodia formation, tight junction disruption, microvilli effacement, or SGLT-1 inactivation.

In other embodiments, an ADAS provided by the invention comprising a bacterial secretion system comprises a T6SS. In some embodiments, the T6SS, in its natural host, targets a bacterium and contains an effector that kills the bacteria. In certain particular embodiments, the T6SS is derived from P. putida K1-T6SS and, optionally, wherein the effector comprises the amino acid sequence of Tke2 (Accession AUZ59427.1), or a functional fragment thereof. In other embodiments, the T6SS, in its natural host, targets a fungi and contains an effector that kills fungi, e.g., the T6SS is derived from Serratia Marcescens and the effectors comprise the amino acid sequences of: Tfe1 (Genbank: SMDB11_RS05530) or Tfe2 (Genbank: SMDB11_RS05390).

In other embodiments of an ADAS provided by the invention that contains a bacterial secretion system, the bacterial secretion system is capable of exporting a cargo extracellularly. In certain more particular embodiments, the bacterial secretion system is T1 SS, T2SS, TSSS, T7SS, Sec, or Tat.

E. ADAS Lacking Proteases, RNases, and/or LPS

In another aspect, the invention provides a composition comprising a plurality of ADAS (e.g., highly active ADAS), wherein the ADAS have a reduced protease level or activity relative to an ADAS produced from a wild-type parent bacterium. In some aspects, the ADAS is produced from a parent bacterium that has been modified to reduce or eliminate expression of at least one protease.

In another aspect, the invention provides a composition comprising a plurality of ADAS (e.g., highly active ADAS), wherein the ADAS have a reduced RNAse level or activity relative to an ADAS produced from a wild-type parent bacterium. In some aspects, the ADAS is produced from a parent bacterium that has been modified to reduce or eliminate expression of at least one RNAse. In some embodiments, the RNase is an endoribonuclease or an exoribonuclease.

In another aspect, the invention provides a composition comprising a plurality of ADAS, wherein the ADAS has been modified to have reduced lipopolysaccharide (LPS). In some embodiments, the modification is a mutation in Lipid A biosynthesis myristoyltransferase (msbB).

In certain embodiments, an ADAS provided by the invention lacks one or more metabolically non-essential proteins. A “metabolically non-essential protein” non-exhaustively includes: fimbriae, flagella, undesired secretion systems, transposases, effectors, phage elements, or their regulatory elements, such as flhC or OmpA. In some embodiments, an ADAS provided by the invention lacks one or more of an RNAse, a protease, or a combination thereof, and, in particular embodiments, lacks one or more endoribonucleases (such as RNAse A, RNAse h, RNAse III, RNAse L, RNAse PhyM) or exoribonucleases (such as RNAse R, RNAse PH, RNAse D); or serine, cysteine, threonine, aspartic, glutamic and metallo-proteases; or a combination of any of the foregoing.

F. ADAS Comprising a Targeting Moiety

In another embodiment, the invention provides a composition comprising a plurality of ADAS, wherein the ADAS comprises a targeting moiety. In some embodiments, the targeting moiety is a nanobody, a carbohydrate binding protein, or a tumor-targeting peptide. In some embodiments, the targeting moiety is an endogenous surface ligand of the parent cell (e.g., a surface ligand that is inherited by the ADAS). In other embodiments, the targeting moiety is an exogenous ligand (e.g., an exogenous tissue targeting ligand) that is added to the ADAS using any of the methods for modifying ADAS described herein. The targeting moiety may promote tissue-associated targeting of the ADAS to a tissue type or cell type.

In certain embodiments, the nanobody is a nanobody directed to a tumor antigen, such as HER2, PSMA, or VEGF-R. In other embodiments, the carbohydrate binding protein is a lectin, e.g. Mannose Binding Lectin (MBL). In still other embodiments, the tumor-targeting peptide is an RGD motif or CendR peptide.

G. ADAS Derived from Commensal or Pathogenic Parent Strains

In another embodiment, the invention provides a composition comprising a plurality of ADAS (e.g., highly active ADAS), wherein the ADAS is derived from a parent bacterium that is a mammalian pathogen or a mammalian commensal bacterium. In some instances, the mammalian commensal bacterium is a Staphylococcus, Bifidobacterium, Micrococcus, Lactobacillus, or Actinomyces species or the mammalian pathogenic bacterium is enterohemorrhagic Escherichia coli (EHEC), Salmonella typhimurium, Shigella flexneri, Yersinia enterolitica, or Helicobacter pylori.

In another embodiment, the invention provides a composition comprising a plurality of ADAS (e.g., highly active ADAS), wherein the ADAS is derived from a parent bacterium that is a plant pathogen or a plant commensal bacterium. In some instances, the plant commensal bacterium is Bacillus subtilis or Pseudomonas putida or the plant pathogenic bacterium is a Xanthomonas species or Pseudomonas syringae.

H. ADAS Derived from Auxotrophic Parent Strains

In another embodiment, the invention provides a composition comprising a plurality of ADAS (e.g., highly active ADAS), wherein the ADAS is derived from an auxotrophic parent bacterium, i.e., a a parent bacterium that is unable to synthesize an organic compound required for growth. Such bacteria are able to grow only when the organic compound is provided.

I. ADAS Comprising Additional Moieties

An ADAS, in certain embodiments, includes a functional ATP synthase and, in some embodiments, a membrane embedded proton pump. ADAS can be derived from different sources including: a parental bacterial strain (“parental strain”) engineered or induced to produce genome-free enclosed membrane systems, a genome-excised bacterium, a bacterial cell preparation extract (e.g., by mechanical or other means), or a total synthesis, optionally including fractions of a bacterial cell preparation. In some embodiments, a highly active ADAS has an ATP synthase concentration of at least: 1 per 10000 nm², 1 per 5000 nm², 1 per 3500 nm², 1 per 1000 nm².

ADAS provided by the invention can include a variety of additional components, including, for example, photovoltaic pumps, retinals and retinal-producing cassettes, metabolic enzymes, targeting agents, cargo, bacterial secretion systems, and transporters, including combinations of the foregoing, including certain particular embodiments described, below. In certain embodiments, the ADAS lack other elements, such as metabolically non-essential genes and/or certain nucleases or proteases.

In certain embodiments, the an ADAS provided by the invention comprises an ATP synthase, optionally lacking a regulatory domain, such as lacking an epsilon domain. Deletion can be accomplished by a variety of means. In certain embodiments, the deletion in by inducible deletion of the native epsilon domain. In certain embodiments, deletion can be accomplished by flanking with LoxP sites and inducible Cre expression or CRISPR knockout, or be inducible (place on plasmid under a tTa tet transactivator in an ATP synthase knockout strain)

An ADAS, in some embodiments, can include a photovoltaic proton pump. In certain embodiments, the photovoltaic proton pump is a proteorhodopsin. In more particular embodiments, the proteorhodopsin comprises the amino acid sequence of proteorhodopsin from the uncultured marine bacterial clade SAR86, GenBank Accession: AAS73014.1. In other embodiments, the photovoltaic proton pump is a gloeobacter rhodopsin. In certain embodiments, the photovoltaic proton pump is a bacteriorhodopsin, deltarhodopsin, or halorhodopsin from Halobium salinarum, Natronomonas pharaonis, Exiguobacterium sibiricum, Haloterrigena turkmenica, or Haloarcula marismortui.

In some embodiments, an ADAS provided by the invention further comprising retinal. In certain embodiments, an ADAS provided by the invention further comprises a retinal synthesizing protein (or protein system), or a nucleic acid encoding the same.

In certain embodiments, an ADAS provided by the invention further comprises one or more glycolysis pathway proteins. In some embodiments, the glycolysis pathway protein is a phosphofructokinase (Pfk-A), e.g., comprising the amino acid sequence of UniProt accession P0A796 or a functional fragment thereof. In other embodiments the glycolysis pathway protein is triosephosphate isomerase (tpi), e.g., comprising the amino acid sequence of UniProt accession P0A858, or a functional fragment thereof.

J. ADAS Compositions and Formulations

The present invention provides compositions or preparations that contain an ADAS provided by the invention, including, inter alia, a highly active ADAS preparation provided by the invention or an ADAS preparation wherein a plurality of individual ADAS lack a cell division topological specificity factor, e.g., lack a minE gene product, and optionally wherein the ADAS preparation is substantially free of viable cells. Collectively, these are “compositions provided by the invention” or “a composition provided by the invention”, or the like and can contain any ADAS provided by the invention and any combination of ADAS provided by the invention.

For example, in some embodiments, a composition provided by the invention contains at least about: 80, 81, 82, 83, 84, 85, 90, 95, 96, 97, 98, 99, 99.1, 99.2, 99.3, 99.4, 99.5, 99.6, 99.7, 99.8, 99.9%, or more ADAS that contain a bacterial secretion system. In particular embodiments, the bacterial secretion system is one of T3SS, T4SS, T3/4SS, or T6SS.

In some embodiments, a composition provided by the invention contains ADAS that contain a T3SS, where the ADAS comprise a mean T3SS membrane density greater than 1 in about: 40000, 35000, 30000, 25000, 19600, 15000, 10000, or 5000 nm². In certain particular embodiments, the ADAS is derived from a S. typhimurium or E. coli parental strain.

Certain embodiments of the compositions provided by the invention contain ADAS that contain a T3SS, where the ADAS comprise a mean T3SS membrane density greater than 1 in about: 300000, 250000, 200000, 150000, 100000, 50000, 20000, 10000, 5000 nm². In certain particular embodiments, the ADAS is derived from an Agrobacterium tumefaciens parental strain.

In another aspect, the invention provide a composition of ADAS, wherein at least about: 80, 81, 82, 83, 84, 85, 90, 95, 96, 97, 98, 99, 99.1, 99.2, 99.3, 99.4, 99.5, 99.6, 99.7, 99.8, 99.9%, or more of the ADAS contain a bacterial secretion system, including T3, T4, T3/4SS, T6SS, and optionally including one or more of: exogenous carbohydrates, phosphate producing synthases, light responsive proteins, import proteins, enzymes, functional cargo, organism-specific effectors, fusion proteins.

As will be readily apparent the compositions and preparations provided by the invention can contain any ADAS provided by the invention, such as highly active ADAS or ADAS that lack a minE gene product.

The compositions provided by the invention can be prepared in any suitable formulation. For example, the formulation can be suitable for IP, IV, IM, oral, topical (cream, gel, ointment, transdermal patch), aerosolized, or nebulized administration. In some embodiments, a formulation is a liquid formulation. In other embodiments, the formulation is a lyophilized formulation.

In some embodiments, an ADAS composition described herein comprises less than 100 colony-forming units (CFU/mL) of viable bacterial cells, e.g., less than 50 CFU/mL, less than 20 CFL/mL, less than 10 CFU/mL, less than 1 CFU/mL, or less than 0.1 CFU/mL of viable bacterial cells.

In some embodiments, the invention provides an ADAS composition wherein the ADAS are lyophilized and reconstituted, and wherein the reconstituted ADAS have an ATP concentration that is at least 90% of the ATP concentration of an ADAS that has not been lyophilized, e.g, at least 95%, 98%, or at least equal to the ATP concentration of an ADAS that has not been lyophilized.

In some embodiments, the invention provides an ADAS composition wherein the ADAS are stored, e.g., stored at 4° C., wherein after storage, the ADAS have an ATP concentration that is at least 90% of the ATP concentration of an ADAS that has not been stored, e.g., at least 95%, 98%, or at least equal to the ATP concentration of an ADAS that has not been stored. In some embodiments, the storage is for at least one day, at least one week, at least two weeks, at least three weeks, at least one month, at least two months, at least six months, or at least a year.

In some embodiments, ADAS may be preserved or otherwise in a “quiescent” state and then rapidly become activated.

In some embodiments, the ADAS composition is formulated for delivery to an animal, e.g., formulated for intraperitoneal, intravenous, intramuscular, oral, topical, aerosolized, or nebulized administration.

In some embodiments, the ADAS composition is formulated for delivery to a plant. In some aspects, the composition includes an adjuvant, e.g., a surfactant (e.g., a nonionic surfactant, a surfactant plus nitrogen source, an organo-silicone surfactant, or a high surfactant oil concentrate), a crop oil concentrate, a vegetable oil concentrate, a modified vegetable oil, a nitrogen source, a deposition (drift control) and/or retention agent (with or without ammonium sulfate and/or defoamer), a compatibility agent, a buffering agent and/or acidifier, a water conditioning agent, a basic blend, a spreader-sticker and/or extender, an adjuvant plus foliar fertilizer, an antifoam agent, a foam marker, a scent, or a tank cleaner and/or neutralizer. In some embodiments, the adjuvant is an adjuvant described in the Compendium of Herbicide Adjuvants (Young et al. (2016). Compendium of Herbicide Adjuvants (13th ed.), Purdue University).

In some embodiments, the ADAS composition is formulated for delivery to an invertebrate, (e.g., arthropod (e.g., insect or arachnid), nematode, protozoan, or annelid). In some embodiments, the ADAS composition is formulated for delivery to an insect.

In some embodiments, the composition is formulated as a liquid, a solid, an aerosol, a paste, a gel, or a gas composition.

K. ADAS Comprising an Enzyme

In one aspect, the invention features a composition comprising a plurality of ADAS, wherein the ADAS comprise an enzyme and wherein the enzyme alters a substrate to produce a target product. In some embodiments, the substrate is present in the ADAS and the target product is produced in the ADAS. In other embodiments, the substrate is present in a target cell or environment to which the ADAS is delivered. In some embodiments, the enzyme is diadenylate cyclase A, the substrate is ATP, and the target product is cyclic-di-AMP.

### IV. Methods of Manufacturing Adas

A. Making ADAS and Highly Active ADAS

In some aspects, the invention features a method for manufacturing a composition comprising a plurality of ADAS, the composition being substantially free of viable bacterial cells, the method comprising (a) making, providing, or obtaining a plurality of parent bacteria having a reduction in the level or activity of a cell division topological specificity factor; (b) exposing the parent bacteria to conditions allowing the formation of a minicell, thereby producing the highly active ADAS; and (c) separating the highly active ADAS from the parent bacteria, thereby producing a composition that is substantially free of viable bacterial cells.

Parent bacteria include any suitable bacterial species from which an ADAS may be generated (e.g., species that may be modified using methods described herein to produce ADAS). Table 1 provides a non-limiting list of suitable genera from which ADAS can be derived.

TABLE 1
Bacterial species for ADAS production

Gram negative bacteria
Escherichia
Acinetobacter
Agrobacterium
Anabaena
Anaplasma
Aquifex
Azoarcus
Azotobacter
Azospirillum
Bartonella
Bordetella
Bradyrhizobium
Brucella
Buchnera
Burkholderia
Candidatus
Chromobacterium
Crocosphaera
Coxiella
Dechloromonas
Desulfitobacterium
Desulfotalea
Erwinia
Francisella
Fusobacterium
Gloeobacter
Gluconobacter
Helicobacter
Legionella
Magnetospirillum
Mesorhizobium
Methylobacterium
Methylococcus
Neisseria
Nitrosomonas
Nostoc
Photobacterium
Photorhabdus
Phyllobacterium
Polaromonas
Prochlorococcus
Pseudomonas
Psychrobacter
Ralstonia
Rhizobium
Rickettsia
Rubrivivax
Salmonella
Shewanella
Shigella
Sinorhizobium
Synechococcus
Synechocystis
Thermosynechococcus
Thermotoga
Thermus
Thiobacillus
Trichodesmium
Vibrio
Wigglesworthia
Wolinella
Xanthomonas
Xylella
Yersinia
Gram positive bacteria
Bacillus
Clostridium
Deinococcus
Exiguobacterium
Geobacillus
Lactobacillus
Listeria
Moorella
Oceanobacillus
Symbiobacterium
Thermoanaerobacter

In some aspects, the invention features methods for manufacturing any of the ADAS compositions, e.g., highly active ADAS compositions, described in Sections III and IV herein. For example, provided herein are methods for making highly active ADAS; methods for making ADAS lacking a cell division topological specificity factor and, optionally, lacking a Z-ring inhibition protein (e.g., methods of making ADAS from ΔminCDE parent bacteria), and methods for making any of the ADAS mentioned herein, wherein the ADAS comprises a cargo.

In some embodiments, the ADAS (e.g., highly active ADAS) is made from a parental strain that is a plant bacterium, such as a plant commensal bacterium (e.g., Bacillus subtilis or Pseudomonas putida), a plant pathogen bacterium (e.g., Xanthomonas sp. or Pseudomonas syringae), or a bacterium that is capable of plant rhizosphere colonization and/or root nodulation, e.g., a Rhizobia bacterium.

In some embodiments, the ADAS (e.g., highly active ADAS) is made from a parental strain that is a symbiont of an invertebrate, e.g., a symbiont of an arthropod (e.g., insect or arachnid), nematode, protozoan, or annelid. In embodiments, the invertebrate is a pest or a pathogen of a plant or of an animal.

In some embodiments, the ADAS (e.g., highly active ADAS) is made from a parental strain that is capable of genetic transformation, e.g., Agrobacterium.

In some embodiments, the ADAS (e.g., highly active ADAS) is made from a parent strain that is a human bacterium, such as a commensal human bacterium (e.g., E. coli, Staphylococcus sp., Bifidobacterium sp., Micrococcus sp., Lactobacillus sp., or Actinomyces sp.) or a pathogenic human bacterium (e.g., Escherichia coli EHEC, Salmonella typhimurium, Shigella flexneri, Yersinia enterolitica, or Helicobacter pylon), or an extremophile.

In some embodiments, the ADAS and/or parent strain is a functionalized derivative of any of the foregoing, for example including a functional cassette, such as a functional cassette that induces the bacterium to do one or more of: secrete antimicrobials, digest plastic, secrete insecticides, survives extreme environments, make nanoparticles, integrate within other organisms, respond to the environment, and create reporter signals.

Parent bacteria may include functionalized derivatives of any of the foregoing, for example including a functional cassette, such as a functional cassette that induces the bacterium to do one or more of: secrete antimicrobials, digest plastic, secrete insecticides, survives extreme environments, make nanoparticles, integrate within other organisms, respond to the environment, and create reporter signals.

In some embodiments, an ADAS is derived from a parental strain engineered or induced to overexpress ATP synthase. In some more particular embodiments, the ATP synthase is heterologous to the parental strain. In certain particular embodiments, the parental strain is modified to express a functional F₀F₁ ATP synthase.

In certain embodiments, an ADAS provided by the invention is obtained from a parental strain cultured under a condition selected from: applied voltage (e.g., 37 mV), non-atmospheric oxygen concentration (e.g., 1-5% 02, 5-10% 02, 10-15% 02, 25-30% 02), low pH (about: 4.5, 5.0, 5.5, 6.0, 6.5), or a combination thereof.

The highly active ADAS of any one of the preceding claims, which is made from an extremophile, including functionalized derivatives of any of the foregoing, for example including a functional cassettes, such as a functional cassette that induces the bacterium to do one or more of: secrete antimicrobials, digest plastic, secrete insecticides, survives extreme environments, make nanoparticles, integrate within other organisms, respond to the environment, and create reporter signals.

Owing to the diversity of bacterium, ADAS can be made with modified membranes, e.g., to improve the biodistribution of the ADAS upon administration to a target cell. In certain embodiments, the membrane is modified to be less immunogenic or immunostimulatory in plants or animals. For example, in certain embodiments, the ADAS is obtained from a parental strain, wherein the immunostimulatory capabilities of the parental strain are reduced or eliminated through post-production treatment with detergents, enzymes, or functionalized with PEG. In certain embodiments, the ADAS is made from a parental strain and the membrane is modified through knockout of LPS synthesis pathways in the parental strain, e.g., by knocking out msbB. In other particular embodiments, the ADAS is made from a parental strain that produces cell wall-deficient particles through exposure to hyperosmotic conditions.

In some embodiments, the methods include transforming a parental strain with an inducible DNAse system, such as the exol (NCBI GeneID: 946529) & sbcD (NCBI GeneID: 945049) nucleases, or the I-CeuI (e.g., Swissprot: P32761.1) nuclease. In more particular embodiments, the methods include using a single, double, triple, or quadruple auxotrophic strain and having the complementary genes on the plasmid encoding the inducible nucleases.

In some embodiments, the methods of the methods provided by the invention, the parental strain is cultured under a condition selected from: applied voltage (e.g., 37 mV), non-atmospheric oxygen concentration (e.g., 1-5% O₂, 5-10% O₂, 10-15% O₂, 25-30% O₂), low pH (4.5-6.5), or a combination thereof.

In certain embodiments, the methods of the methods provided by the invention, the parental strain lacks flagella and undesired secretion systems, optionally where the flagella and undesired secretion systems are removed using lambda red recombineering.

In some embodiments, the methods of provided by the invention, flagella control components are excised from the parental strain genome via, for example, insertion of a plasmid containing a CRISPR domain that is targeted towards flagella control genes, such as flhD and flhC.

In certain embodiments, the methods provided by the invention are for making a highly active ADAS, where an ADAS comprising a plasmid containing a rhodopsin-encoding gene is cultured in the presence of light. In more particular embodiments, the rhodopsin is proteorhodopsin from SAR86 uncultured bacteria, having the amino acid sequence of GenBank Accession: AAS73014.1, or a functional fragment thereof. In still more particular embodiments, the culture is supplemented with retinal. In other more particular embodiments, the rhodopsin is proteorhodopsin and the plasmid additionally contains genes synthesizing retinal (such a plasmid is the pACYC-RDS plasmid from Kim et al., Microb Cell Fact, 2012).

In certain particular embodiments, the parental strain contains a nucleic acid sequence encoding a nanobody that is then expressed on the membrane of the ADAS.

In some embodiments of the methods provided by the invention, the parental strain contains a nucleic acid sequence encoding one or more bacterial secretion system operons. Exemplary plasmids include the Salmonella SPI-1 T3SS, the Shigella flexneri T3SS, the Agro Ti plasmid, and the P. putida K1-T655 system.

In certain embodiments, the parental strain comprises a cargo. In some embodiments, the parent strain contains a nucleic acid sequence encoding a set of genes that synthesize a small molecule cargo.

### V. Methods of Using ADAS

A. Methods of Delivering an ADAS

In one aspect, the invention features a method for delivering an ADAS to a target cell, the method comprising (a) providing a composition comprising a plurality of ADAS; and (b) contacting the target cell with the composition of step (a).

In another aspect, the invention features a method for delivering an ADAS to a target cell, the method comprising: (a) providing a composition comprising a plurality of ADAS; and (b) contacting the target cell with the composition of step (a).

The target cell may be, e.g., an animal cell, a plant cell, or an insect cell.

B. Methods of Delivering a Cargo

In another aspect, the invention features a method for delivering a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)) to a target cell, the method comprising: (a) providing a composition comprising a plurality of achromosomal dynamic active systems (ADAS); and (b) contacting the target cell with the composition of step (a).

In another aspect, the invention features a method for delivering a cargo (e.g., a nucleic acid, a plasmid, a polypeptide, a protein, an enzyme, an amino acid, a small molecule, a gene editing system, a hormone, an immune modulator, a carbohydrate, a lipid, an organic particle, an inorganic particle, or a ribonucleoprotein complex (RNP)) to a target cell, the method comprising: (a) providing a composition comprising a plurality of ADAS; and (b) contacting the target cell with the composition of step (a).

The target cell to which the cargo is delivered may be, e.g., an animal cell, a plant cell, or an insect cell.

C. Methods of Modulating a State of a Cell

In one aspect, the invention features a method of modulating a state of an animal cell, the method comprising: (a) providing a composition comprising a plurality of achromosomal dynamic active systems (ADAS); and (b) contacting the animal cell with the composition of step (a), whereby a state of the animal cell is modulated.

In another aspect, the invention features a method of modulating a state of a plant cell, the method comprising: (a) providing a composition comprising a plurality of achromosomal dynamic active systems (ADAS; and (b) contacting the plant cell with the composition of step (a), whereby a state of the plant cell is modulated.

In another aspect, the invention features a method of modulating a state of an insect cell, the method comprising: (a) providing a composition comprising a plurality of achromosomal dynamic active systems (ADAS); and (b) contacting the insect cell with the composition of step (a), whereby a state of the insect cell is modulated.

The modulating may be any observable change in the state (e.g., the transcriptome, proteome, epigenome, biological effect, or health or disease state) of the cell (e.g., an animal, plant, or insect cell) as measured using techniques and methods known in the art for such a measurement, e.g., methods to measure the level or expression of a protein, a transcript, an epigenetic mark, or to measure the increase or reduction of activity of a biological pathway. In some embodiments, modulating the state of the cell involves increasing a parameter (e.g., the level or expression of a protein, a transcript, or activity of a biological pathway) of the cell. In other embodiments, modulating the state of involves decreasing a parameter (e.g., the level or expression of a protein, a transcript, or activity of a biological pathway) of the cell.

D. Methods of Treating an Animal, a Plant, or an Insect

In some aspects, the invention features a method of treating an animal in need thereof, the method comprising (a) providing a composition comprising a plurality of achromosomal dynamic active systems (ADAS); and (b) contacting the animal with an effective amount of the composition of step (a), thereby treating the animal.

The animal in need of treatment may have a disease, e.g., a cancer. In some embodiments, the ADAS carries a chemotherapy cargo or an immunotherapy cargo.

In some aspects, the invention features a method of treating a plant in need thereof, the method comprising (a) providing a composition comprising a plurality of achromosomal dynamic active systems (ADAS); and (b) contacting the plant or a pest (e.g., an insect pest) thereof with an effective amount of the composition of step (a), thereby treating the plant.

In an additional aspect, the invention provides methods of modulating a target cell. The target cell can be any cell, including an animal cell (e.g., including humans and non-human animals, including farm or domestic animals, pests), a plant cell (including from a crop or a pest), a fungal cell, or a bacterial cell. The cell may be isolated, e.g., in vitro or, in other embodiments, within an organism, in vivo. These methods entail providing an ADAS provided by the invention or a composition provided by the invention with access to the target cell, in an effective amount. The access to the target cell may either be direct, e.g., where the target cell is modulated directly by the ADAS, such as by proximate secretion of some agent proximate to the target cell or injection of the agent into the target cell, or indirect. The indirect modulation of the target cell can be by targeting a different cell, for example, by modulating a cell adjacent to the target cell, which adjacent cell may be commensal or pathogenic to the target cell. The adjacent cell, like the target cell may be either in vitro or in vivo—i.e., in an organism, which may be commensal or pathogenic. These methods are collectively “methods of use provided by the invention” and the like. In a related aspect, the invention provides target uses of the ADAS and compositions provided by the invention, consonant with the methods of use provided by the invention.

For example, in some embodiments, the invention provides method of modulating a state of an animal cell, by providing an effective amount of an ADAS provided by the invention or composition provided by the invention access to the animal cell. In certain embodiments, the ADAS or composition is provided access to the animal cell in vivo, in an animal, such as a mammal, such as a human. In some embodiments, the animal cell is exposed to bacteria in a healthy animal. In more particular embodiments, the animal cell is lung epithelium, an immune cell, skin cell, oral epithelial cell, gut epithelial cell, reproductive tract epithelial cell, or urinary tract cell. In still more particular embodiments, the animal cell is a gut epithelial cell, such as a gut epithelial cell from a human subject with an inflammatory bowel disease, such as Crohn's disease or colitis. In yet more particular embodiments, the animal cell is a gut epithelial cell from a subject with an inflammatory bowel disease, and the ADAS comprises a bacterial secretion system and a cargo comprising an anti-inflammatory agent, e.g., an antibody or antibody fragment targeting tumor necrosis factor (TNF) (e.g., an anti-TNF antibody); an antibody or antibody fragment targeting IL-12 (e.g., an anti-IL-12 antibody); or an antibody or antibody fragment targeting IL-23 (e.g., an anti-IL-23 antibody).

In other embodiments the animal cell is exposed to bacteria in a diseased state. In certain embodiments, the animal cell is pathogenic, such as a tumor. In other embodiments, the animal cell is exposed to bacteria in a diseased state, such as a wound, an ulcer, a tumor, or an inflammatory disorder

In certain embodiments, the ADAS is derived from an animal commensal parental strain. In other embodiments, the ADAS is derived from animal pathogenic parental strain.

In certain particular embodiments, the animal cell is contacted to an effective amount of an ADAS comprising a T3/4SS or T6SS and a cargo, wherein the cargo is delivered into the animal cell. In some particular embodiments, the animal cell is provided access to an effective amount of an ADAS comprising a cargo and a secretion system, wherein the cargo is secreted extracellularly and contacts the animal cell.

In some embodiments, the state of the animal cell is modulated by providing an effective amount of an ADAS provided by the invention or a composition provided by the invention with access to a bacterial or fungal cell in the vicinity of the animal cell. That is, these methods entail indirectly modulating the state of the animal cell. In certain embodiments, the bacterial or fungal cell is pathogenic. In more particular embodiments, the fitness of the pathogenic bacterial or fungal cell is reduced. In other certain embodiments, the bacterial or fungal cell is commensal. In more particular embodiments, the fitness of the commensal bacterial or fungal cell is increased. In still more particular embodiments, the fitness of the commensal bacterial or fungal strain is increased via reduction in fitness of number of a competing bacteria or fungi, which may be neutral, commensal, or pathogenic.

In certain particular embodiments, the bacterial or fungal cell in the vicinity of the animal cell is contacted to an effective amount of ADAS comprising a T3/4SS or T6SS and a cargo, wherein the cargo is delivered into the bacterial or fungal cell. In other particular embodiments, the bacterial or fungal cell in the vicinity of the animal cell is provided access to an effective amount of ADAS secreting cargo extracellularly that contacts the bacterial or fungal cell.

In certain embodiments, the ADAS is derived from a parental strain that is a competitor of the bacterial or fungal cell. In other embodiments, the ADAS is derived from a from a parental strain that is mutualistic bacteria of the bacterial or fungal cell.

As will be appreciated, the various method of use provided by the invention that modulate the state of an animal cell can readily be adapted to corresponding methods for modulating the state of a plant, fungal, or bacterial cell. For illustrative purposes, methods for modulating the cell of a plant or fungal cell will be recited more particularly.

Accordingly, in a related aspect the invention provide methods of modulating a state of a plant or fungal cell by providing an effective amount of an ADAS provided by the invention or composition provided by the invention access to: a) the plant or fungal cell, b) an adjacent bacterial or adjacent fungal cell in the vicinity of the plant or fungal cell, or c) an invertebrate, (e.g., arthropod (e.g., insect or arachnid), nematode, protozoan, or annelid) cell in the vicinity of the plant or fungal cell.

In certain embodiments, the ADAS is provided access to the plant cell in planta, e.g., in a crop plant such as row crops, including corn, wheat, soybean, and rice, and vegetable crops including Solanaceae, such as tomatoes and peppers; cucurbits, such as melons and cucumbers; Brassicas, such as cabbages and broccoli; leafy greens, such as kale and lettuce; roots and tubers, such as potatoes and carrots; large seeded vegetables, such as beans and corn; and mushrooms. In some embodiments, the plant or fungal cell is exposed to bacteria in a healthy plant or fungus. In other embodiments, the plant or fungal cell is exposed to bacteria in a diseased state.

In certain embodiments, the plant or fungal cell is dividing, such as a meristem cell, or is pathogenic, such as a tumor. In some embodiments, the plant or fungal cell is exposed to bacteria in a diseased state, such as a wound, or wherein the plant or fungal cell is not part of a human foodstuff.

For certain embodiments the ADAS is derived from a commensal parental strain. In other embodiments, the ADAS is derived from a plant or fungal pathogenic parental strain.

In some embodiments, the ADAS comprises an T3/4SS or T6SS and a cargo, and the cargo is delivered into the plant or fungal cell. In other embodiments, the plant or fungal cell is provided access to an effective amount of an ADAS comprising a bacterial secretion system and a cargo, wherein the bacterial secretion system secretes the cargo extracellularly, thereby contacting the plant or fungal cell with the cargo.

In some embodiments, the methods entail providing an effective amount of an ADAS or composition access to the adjacent bacterial or adjacent fungal cell in the vicinity of the plant or fungal cell. In more particular embodiments, the adjacent bacterial or adjacent fungal cell is pathogenic, optionally wherein the fitness of the pathogenic adjacent bacterial or adjacent fungal cell is reduced. In other more particular embodiments, the adjacent bacterial or adjacent fungal cell is commensal, optionally wherein the fitness of the commensal adjacent bacterial or adjacent fungal cell is increased. In still more particular embodiments, the fitness is increased via reduction of a competing bacteria or competing fungi, which may be neutral, commensal, or pathogenic.

In some embodiments, the adjacent bacterial or adjacent fungal cell is contacted with an effective amount of ADAS comprising a T3/4SS or T6SS and a cargo, wherein the cargo is delivered into the adjacent bacterial or adjacent fungal cell.

In other embodiments, the adjacent bacterial or adjacent fungal cell is provided access to an effective amount of ADAS comprising a bacterial secretion system and a cargo, wherein the bacterial secretion system secretes the cargo extracellularly, thereby contacting the adjacent bacterial or adjacent fungal cell with the cargo.

In some embodiments, the ADAS is derived from a parental strain that is a competitor of the adjacent bacterial or adjacent fungal cells. In other embodiments, the ADAS is derived from a parental strain that is a mutualistic bacterium of the adjacent bacterial or adjacent fungal cell.

In certain embodiments, the methods include providing an effective amount of the ADAS or composition access to an invertebrate, (e.g., arthropod (e.g., insect or arachnid), nematode, protozoan, or annelid) cell in the vicinity of the plant or fungus. In more particular embodiments, the invertebrate is pathogenic. In still more particular embodiments, the fitness of the pathogenic invertebrate cell is reduced. In yet more particular embodiments, the fitness of the pathogenic invertebrate cell is reduced via modulation of symbiotes in the invertebrate cell. In other particular embodiments, the invertebrate is commensal. In more particular embodiments, the fitness of the commensal invertebrate cell is increased. In still more particular embodiments, the fitness is increased via reduction of a competing bacteria or fungi, which may be neutral, commensal, or pathogenic.

In yet another aspect, the invention provide methods of removing one or more undesirable materials from an environment comprising contacting the environment with an effective amount of an ADAS provided by the invention or composition provided by the invention, wherein the ADAS comprises one or more molecules (such as proteins, polymers, nanoparticles, binding agents, or a combination thereof) that take up, chelate, or degrade the one or more undesirable materials. “Environments” are defined as targets that are not cells, such as the ocean, soil, superfund sites, skin, ponds, the gut lumen, and food in a container.

In certain embodiments, the undesirable material includes a heavy metal, such as mercury, and the ADAS includes one or more molecules (such as proteins, polymers, nanoparticles, binding agents, or a combination thereof) that bind heavy metals, such as MerR for mercury. In some embodiments, the undesirable material includes a plastic, such as PET, and the ADAS includes one or more plastic degrading enzymes, such as PETase. In certain embodiments, the undesirable material comprises one or more small organic molecules and the ADAS comprise one or more enzymes capable of metabolizing said one or more small organic molecules.

E. RNA Delivery Methods

In another aspect, the invention provides a composition containing a bacterium or ADAS provided by the invention, wherein the bacterium or ADAS includes a T4SS, an RNA binding protein cargo, and an RNA cargo that is bound by the RNA binding protein and is suitable for delivery into a target cell through the T4SS. In certain embodiments, the RNA binding protein is a Cas9 fused to VirE2 and VirF, the RNA cargo is a guide RNA, and, optionally, the T4SS is the Ti system from Agrobacterium. In other embodiments, the RNA binding protein is p19 from Carnation Italian Ringspot Virus fused to VirE2 or VirF, the RNA cargo is an siRNA, and optionally wherein the T4SS is the Ti system from Agrobacterium.

In a related aspect, the invention provides methods of making these particular compositions, such methods entailing transfecting a plasmid containing the Cas9 fused to VirE2 and VirF and RNA cargo into an Agrobacterium cell.

In a further related aspect, the invention provides methods for delivering RNA to a plant cell or animal cell comprising contacting said plant cell or animal cell with a bacterium or ADAS, wherein the bacteria or ADAS comprises a T4SS, an RNA binding protein cargo, and an RNA cargo, wherein the RNA is delivered to the plant cell or animal cell. In more particular embodiments, the RNA-binding protein cargo is also delivered to the plant cell or animal cell.

### EXAMPLES

Table of Contents

| | |
|---|---|
| Example 1 | Production of ADAS by genetic manipulation |
| Example 2 | Characterization of ADAS |
| Example 3 | Comparison of standard and optimized processes for selective |
| | depletion of viable ADAS parents |

### Example 1: Production of ADAS by Genetic Manipulation

ADAS may be produced from parent bacterial cells by several means. In this example, ADAS are produced by disruption of one or more genes involved in regulating parent cell partitioning functions (i.e., disruption of a z-ring inhibition protein (e.g., ΔminC or ΔminD) or disruption of z-ring inhibition proteins and a cell division topological specificity factor (e.g., ΔminCDE) or overproduction of the FtsZ and/or associated division machinery) or production of DNA nucleases that digest the genetic material of the cell. This example details genetic means of creating ADAS-producing strains via disruption of the min operon, over-expression of the septum machinery component FtsZ or destruction of existing genetic material via expression of a nuclease.

A. Production of ADAS Via Min Mutations

To disrupt the min operon, Lambda-RED recombineering methodology was adopted according to protocols laid out in Datsenko and Wanner, PNAS, 97(12): 6640-6645, 2000. Strains for engineering and containing the plasmids for the Lambda-RED system were acquired from the Coli Genetic Stock Center (CGSC) at Yale University. Briefly, primers were designed to nonpolarly delete the coding sequences of E. coli minC (generating the parent bacterial strain MACH061), minD (generating the parent bacterial strain MACH062), or the entire minCDE operon (generating the parent bacterial strain MACH060) by encoding approximately 40 base pairs of genomic homology into the 5′ ends of primers. The 3′ ends of these primers are homologous to plasmids pKD3 and pKD4 of the Lambda-RED system, which provide antibiotic markers that were used to select for parent bacterial strains inheriting the target mutations. Primer sequences used for deletion are provided in Table 2. After performing standard PCR using the primers with pKD3 as a DNA template, the purified amplicon was transformed via electroporation into bacteria prepared with pKD46, the plasmid containing the phage-derived Lambda-RED homologous recombination system, according to the methods of Datsenko and Wanner, PNAS, 97(12): 6640-6645, 2000. Transformants were selected on LB agar with 35 μg/mL chloramphenicol. These resulting colonies were confirmed to have the genetic disruption (i.e., ΔminC, ΔminD, or ΔminCDE) using standard allele-specific PCR. Strain genotypes are provided in Table 3.

B. Production of ADAS by Overexpression of ftsZ

To create ADAS from the overexpression of septum machinery, we constructed a plasmid that drives expression of the FtsZ Z-ring protein from wild-type E. coli. In brief, a strong ribosome binding site and the coding sequence for the E. coli FtsZ protein were de novo optimized using computational tools from De Novo DNA. This translational unit was ordered for de novo DNA synthesis from Integrated DNA Technologies (IDT™) and cloned into a backbone using standard cloning techniques. The resulting plasmid, pFtsZ (Table 4), features a TetR repressor, a TetA promoter that is repressed by the TetR protein, a kanamycin resistance marker, and a pMB1 origin of replication. When transformed into a compatible bacterium, pFtsz can be induced to overproduce the FtsZ protein via addition of anhydrotetracycline to the culture. This protein is then capable of forming spontaneous protofilaments, which cause asymmetric division of parent bacterial cells and, thereby, ADAS production.

C. Production of Genome-Deleted ADAS by Genetic Manipulation

In this example, ADAS are produced using a nuclease which degrades the genetic material within a bacterial cell. Expression and/or activity of the nuclease may be inducible. The expression of said nuclease causes ADAS to be produced uniformly throughout a bacterial population.

TABLE 2
Primers

| Primer Name | Description | Sequence |
|---|---|---|
| oAF75 | KO MinCDE FWD | SEQ ID NO: 10 |
| oAF76 | KO MinCDE REV | SEQ ID NO: 11 |
| oAF77 | KO MinC FWD | SEQ ID NO: 12 |
| oAF78 | KO MinC REV | SEQ ID NO: 13 |
| oAF79 | KO MinD FWD | SEQ ID NO: 14 |
| oAF80 | KO MinD REV | SEQ ID NO: 15 |
| oAF167 | KO SalTy MinCDE FWD | SEQ ID NO: 16 |
| oAF168 | KO SalTy MinCDE REV | SEQ ID NO: 17 |
| oDM17 | EcoRI T261I fragment | SEQ ID NO: 5 |
| | FOR | |
| oDM18 | EcoRI R56Q fragment | SEQ ID NO: 6 |
| | FOR | |
| oDM19 | EcoRI R56Q fragment | SEQ ID NO: 7 |
| | REV | |
| oDM20 | EcoRI T261I fragment | SEQ ID NO: 8 |
| | REV | |

TABLE 3
Strains

| Name | Description | Genotype | Alias |
|---|---|---|---|
| MACH009 | BW25113 | Δ(araD-araB)567, ΔlacZ4787(::rrnB-3), λ-, | CGSC 7636 |
| | | rph-1, Δ(rhaD-rhaB)568, hsdR514 | |
| MACH060 | MACH009 ΔminCDE::CamR | BW25113 ΔminCDE::CamR | This work |
| MACH065 | MG1655 | F-, λ-, rph-1 | CGSC 7740 |
| MACH178 | MACH009 pFtsZ | BW25113 pFtsZ | This work |
| MACH200 | MACH065 ΔminCDE::CamR | MG1655 ΔminCDE::CamR | This work |
| MACH289 | Salmonella typhimurium | Salmonella typhimurium LT2 | This work |
| | Δmin | ΔminCDE::CamR | |

TABLE 4
Plasmids

| Plasmid | | | |
|---|---|---|---|
| Name | Description | Sequence | Reference |
| pFtsZ | pMB1 KanR TetR-PtetA-FtsZ | SEQ ID NO: 18 | This work |

### Example 2: Characterization of ADAS

This example describes methods of characterizing purified populations of ADAS and/or unpurified ADAS-producing bacterial cultures using a variety of approaches, such as nanoparticle characterization and viable cell plating.

A. Viable Cell Plating

To determine the concentration of viable parent bacterial cells present before and after purification, the ADAS-producing cultures and the purified ADAS populations described in Example 3 are assayed via viable cell plating. Ten microliters of each of several serial dilutions is spotted on selective media and incubated at 37° C. to allow the growth of viable cells. After 24 hours, dilutions containing 1-100 colonies are counted to enumerate the number of colony forming units (CFU) per mL of sample.

B. Nanoparticle Characterization

A purified population of ADAS from Example 3 is suspended in 1×PBS, diluted to a concentration between 10⁷ and 10⁹ particles per mL, and TWEEN® 20 (Sigma Aldrich) is added to a final concentration of 0.1% (v/v) to minimize particle aggregation. This suspension of ADAS is diluted 20-fold and loaded onto a CS2000 cartridge (Technology® Supplies Ltd.), and analysis is performed on a Spectradyne® nCS1™ Nanoparticle Analyzer. Additionally, quantification of small molecules (e.g., ATP), nucleic acids, or proteins (e.g., GFP) observed within a purified population of ADAS can be divided by the aggregate volume of ADAS or the number of particles present in the assay, enabling calculation of the average concentration of small molecules, nucleic acids, or proteins of interest within a purified population of ADAS.

### Example 3: Comparison of Standard and Optimized Processes for Selective Depletion of Viable ADAS Parents

This example describes an optimized method for purifying populations of ADAS from a culture of an ADAS-producing bacterial parent strain, which may be compared to a method using traditional selection measures. Purification separates ADAS from contaminating viable parent bacterial cells, which may be larger and contain a genome. This method may be employed to purify ADAS-containing preparations from any ADAS-producing strain, including any strain described herein, for example, the strains of Table 4.

In the traditional method, ADAS were purified from high cell density cultures of ADAS-producing strains via combinations of 1) low speed centrifugation, 2) selective outgrowth, and 3) buffer exchange/concentration. Low-speed centrifugation procedures were used to selectively deplete viable parent bacterial cells and large cellular debris, while retaining enriching ADAS in a mixed suspension of the supernatant. However, due to the degree of size similarity, selective outgrowth procedures were employed to reduce the number of viable parent bacterial cells retained after the low speed separation. Selection was performed by the addition of compounds that are directly anti-microbial (i.e., toxic to cells having a microbial genome) and/or compounds that enhance viable cell sedimentation via low speed centrifugation. Buffer exchange/concentration procedures were used to transition ADAS from larger volumes of bacterial culture media into smaller volumes of 1×PBS while removing culture additives and cellular debris.

The optimized method described herein differs by the inclusion of a concentration and buffer exchange step following low-speed centrifugation, but prior to the selective outgrowth conditions. This step permits the concentration of both residual parent cells and ADAS into a more manageable volume that minimizes the usage of selective agents, which are often expensive. Additionally, including an exchange for fresh media in the selective outgrowth procedure can have significant impact on the efficacy of antibiotic treatment. Without being bound by theory, cells grown to stationary phase where the yield of ADAS is highest enter a sort of “senescence” (e.g., stringent response) that can enable resistance to growth-dependent antibiotics, and replenishing nutrients available to the residual parent bacterial cells causes them to enter a metabolic state that permits killing via antibiotic selection (e.g., a metabolic state involving growth).

An ADAS-producing strain, MACH060 (Table 3), was generated using the molecular cloning procedures described in Example 1, then cultured to high cell density in culture medium. Cultures may be scaled up, e.g., from 1 mL to 1000 mL or more culture medium. In this example, two 1 L cultures were grown overnight and combined into a single 2 L culture. A 1 mL aliquot was removed, serially diluted, and plated to determine starting viable parent burden, as described in Example 2.

From this pooled 2 L culture, 1 L was transferred to each of two centrifuge tubes and subjected to a low-speed centrifugation procedure aimed at pelleting intact cells and large cell debris while maintaining ADAS in the supernatant. Low-speed centrifugation procedures were performed at 4° C. for 40 minutes at 4,000×g in a Sorvall™ Lynx 6000 Superspeed Centrifuge (Thermo Scientific™) in which the rate of rotor acceleration was set to the lowest possible setting.

Following low-speed centrifugation, 1 mL aliquots were removed from each tube and plated for residual viable cell burden. One of the two centrifuge tubes was decanted into an appropriate Erlenmeyer flask and supplemented with 100 μg/mL ceftriaxone, a beta-lactam antibiotic, and the pellet was discarded. This flask, which represents the nonoptimized process, was incubated at 37° C. with shaking at 250 RPM for 1 hour and then transferred to 4° C. until further processing.

The other centrifuge tube, which represents the optimized selective process, was decanted into fresh centrifuge vessels and centrifuged at 17,000×g for 60 minutes on the fastest ramp speed. The supernatant was aspirated and discarded and the pellet, which contained ADAS and residual contaminating viable parents, was resuspended in 50 mL of fresh media supplemented with 100 μg/mL ceftriaxone. Optimized selective outgrowth was performed at 37° C. with shaking at 250 RPM for 1 hour, and then the preparation was transferred to 4° C. until further processing.

ADAS preparations were then transferred to sterile centrifuge tubes and subjected to an additional round of low-speed centrifugation for 15 minutes at 4° C., 4,000×g. The supernatant, representing the purified ADAS, was collected at 20,000×g for 20 minutes, the pellet was washed with fresh 1×PBS, recollected at 20,000×g for 20 minutes and, finally, resuspended in 10 mL of fresh 1×PBS. The purified ADAS were evaluated for parent burden via serial dilution and plating and assessed for particle concentration as described in Example 2.

The results from the direct comparison of the standard ADAS process to the optimized process disclosed within are displayed in Table 5. The results show very similar or equivalent parent burden in the steps preceding the optimized selection process and a similar yield of ADAS between both process methods. However, parent burden is reduced >3000-fold in the optimized method versus the standard method. This increase in purity was achieved while reducing the use of selective agent (i.e., ceftriaxone) 20-fold.

TABLE 5
Comparison of parent burden in standard and optimized selection processes

| | | | | | Fold |
|---|---|---|---|---|---|
| | Parent | Parent | | ADAS | reduction |
| | burden in | burden after | Parent | concentration | in parent |
| | original | first slow | burden in | in final prep | burden from |
| | culture | spin | final prep | (Particles/mL | original |
| Sample | (CFU/mL) | (CFU/mL) | (CFU/mL) | [250-2000 nm]) | culture |
| Standard | 3.4 × 10⁹ | 1.4 × 10⁶ | 5.6 × 10⁶ | 1.2 × 10⁹ | 6.07 × 10⁴ |
| Process | | | | | |
| Optimized | 3.4 × 10⁹ | 1.3 × 10⁶ | 1.8 × 10³ | 7.1 × 10⁸ | 1.89 × 10⁸ |
| Process | | | | | |

| | |
|---|---|
| Fold improvement of Optimized Process | 3.11 × 10³ |

Levels of selective agent residues in final ADAS preparations were assessed using LC-MS/MS. Briefly, a known quantity of ADAS in solution is assessed for the presence and concentration of a selective agent (e.g., carbenicillin, ceftriaxone, or ciprofloxacin). An aliquot of ADAS solution is diluted with water and disrupted by sonication. Samples are centrifuged, and the supernatant is transferred to an HPLC vial. Final determination is made by high performance liquid chromatography with triple quadrupole mass spectrometric detection (LC-MS/MS) using electrospray ionization (ESI) in positive ionization mode. The limit of quantification is 0.01 ppm (10 ng/mL).

Residue levels of the selective agent in the final product were almost non-detectable or were non-detectable. In one example using ceftriaxone, residue levels were 4.11 ng/ml (reduced from an initial concentration of 5370 ng/ml). In another example using ciprofloxacin, residue levels were 0 ng/ml (reduced from an initial concentration of 265 ng/mL).

Detection was performed as follows: Column: Sunfire C18 column; 2.1*150 mm; Mobile Phases: A: 0.1% formic acid (FA) in water, B: 0.1% FA in Acetonitrile; Detection: LC-MS-MS yMax: Carb: 254, CIP:222 and CEP:278; Injection volume: 50 uL. Conditions for liquid chromatography (LC) are shown in Table 6.

TABLE 6
Liquid chromatography (LC) conditions

| Time | % A | % B |
|---|---|---|
| 0 | 95 | 5 |
| 5 | 5 | 95 |
| 6 | 5 | 95 |
| 6.1 | 95 | 5 |
| 10 | 95 | 5 |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

Other embodiments are within the claims.

What is claimed is:

1. A method for producing an achromosomal dynamic active system (ADAS) preparation, the method comprising:
(a) providing a preparation comprising a plurality of ADAS and a plurality of parent bacterial cells; and
(b) exposing the preparation to a culture medium and a growth-selective agent under growth-promoting conditions for the parent bacterial cells, wherein the growth-selective agent reduces viability or inhibits cell division of the growing parent bacterial cells, thereby producing an ADAS preparation that is substantially enriched in ADAS.

2. The method of claim 1, wherein the preparation of step (a) has been concentrated relative to a culture from which the plurality of ADAS and plurality of parent bacterial cells are derived.

3. The method of claim 1, wherein the growth-selective agent is an agent that is toxic to parent bacterial cells.

4. The method of claim 3, wherein the agent that is toxic to parent bacterial cells is an antibiotic.

5. The method of claim 4, wherein the antibiotic is a beta lactam.

6. The method of claim 4, wherein the antibiotic is ceftriaxone, kanamycin, carbenicillin, gentamicin, or ciprofloxacin.

7. The method of claim 3, wherein the agent that is toxic to parent bacterial cells is a chemical.

8. The method of claim 7, wherein the chemical is sodium chloride, sodium hydroxide, M hydrochloric acid, glucose, a plurality of cas-amino acids, or a plurality of D-amino acids.

9. The method of claim 1, wherein the growth-selective agent is an agent that increases the sensitivity to sedimentation of parent bacterial cells.

10. The method of claim 9, wherein the growth-selective agent induces a filamentous morphology in parent bacterial cells.

11. The method of claim 9 or 10, wherein the sedimentation is performed by low-speed centrifugation.

12. The method of claim 1, wherein the growth-selective agent is an agent that interferes with growth of a bacterial cell wall.

13. The method of any one of claims 1-12, wherein step (b) further comprises providing an agent that promotes the growth of parent bacterial cells.

14. The method of any one of claims 1-13, wherein the exposing comprises incubating the preparation for at least one hour.

15. The method of claim 14, wherein the incubating is performed at a temperature of between 4° C. and 42° C.

16. The method of any one of claims 1-15, wherein the exposure to the culture medium precedes the exposure to the growth-selective agent.

17. The method of claim 1, wherein the preparation of step (a) has been concentrated by at least 20-fold, at least 50-fold, or at least 100-fold.

18. The method of any one of claims 1-17, wherein the preparation of step (a) is a pellet produced by a process comprising providing a supernatant of a culture comprising a plurality of ADAS and a plurality of parent bacterial cells, wherein the supernatant is produced by low-speed centrifugation of the culture, and subjecting the supernatant to high-speed centrifugation, thereby producing the pellet.

19. The method of any one of claims 1-18, wherein step (b) comprises resuspending the pellet in the culture medium.

20. The method of any one of claims 1-19, wherein the parent bacterial cells are derived from a culture at a stationary phase of growth.

21. The method of claim 20, wherein the parent bacterial cells are senescent.

22. The method of any one of claims 16-21, wherein the culture from which the plurality of ADAS and plurality of parent bacterial cells are derived has a volume of at least 1 L.

23. The method of claim 22, wherein the culture has a volume of at least 100 L.

24. The method of any one of claims 1-23, wherein the ADAS are derived from the parent bacterial cells.

25. The method of any one of claims 1-24, further comprising subjecting the ADAS preparation of step (b) to low-speed centrifugation, wherein the supernatant comprises the ADAS preparation.

26. The method of any one of claims 1-25, wherein the ADAS preparation is substantially free of parent bacterial cells.

27. The method of any one of claims 1-26, further comprising concentrating the substantially enriched ADAS preparation.

28. The method of any one of claims 1-26, wherein the method does not comprise contacting the parent cells with a nuclease.

29. An achromosomal dynamic active system (ADAS) preparation produced by the method of any of claims 1-28, wherein the ratio of ADAS to parent cells in the preparation is greater than at least one of 1,000:1, 10,000:1, 100,000:1, 500,000:1, and 1,000,000:1.

30. The ADAS preparation of claim 29, wherein the growth-selective agent is present at a level less than at least one of 80 ng/ml, 70 ng/ml, 60 ng/ml, 50 ng/ml, 40, ng/ml, 30 ng/ml, 20 ng/ml, 10 ng/ml, 5 ng/ml, and 1 ng/ml following step (b) of the method.
